(19) United States
(12) Patent Application Publication
(10) Pub. No.: US 2024/0016418 A1
(43) Pub. Date: Jan. 18, 2024

(54) ELECTROCHEMICAL DEVICES AND METHODS FOR ACCURATE DETERMINATION OF ANALYTE

(71) Applicant: Cercacor Laboratories, Inc., Irvine, CA (US)

(72) Inventors: Anando Devadoss, Irvine, CA (US); Venkatramanan Krishnamani, Irvine, CA (US); Qiurong Shi, Irvine, CA (US); David Shimomoto, Irvine, CA (US); Ngoc Nhu Ly, Irvine, CA (US)

(21) Appl. No.: 18/352,742

(22) Filed: Jul. 14, 2023

Related U.S. Application Data
(60) Provisional application No. 63/487,352, filed on Feb. 28, 2023.
(60) Provisional application No. 63/368,754, filed on Jul. 18, 2022.

(51) Int. Cl. A61B 5/145; A61B 5/1486

(52) U.S. Cl. CPC A61B 5/14532; A61B 5/1486

(57) ABSTRACT

The present disclosure provides devices, systems, and methods capable of optimizing in vivo electrochemical measurement of a molecule of interest, for example glucose. Disclosed aspects may include an interference zapping layer, to which an electric potential may be applied to prevent or minimize various interfering molecules from reaching a working electrode. Aspects of the present disclosure may additionally or alternatively include a sensor including multiple working electrodes. The sensors having multiple working electrodes may measure background current during sensing in order to determine background current specific to each electrode, current due to electrochemical interferents, and current due to the molecule of interest. Sensors may also be capable of executing a measurement mode whereby a plurality of currents are measured over a range of interference zapping layer potentials and a range of working electrode potentials.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Application No. 63/368,754, filed Jul. 18, 2022, and U.S. Provisional Application No. 63/487,352, filed Feb. 28, 2023, the entire contents of which are incorporated by reference herein in their entireties for all purposes and form a part of this specification.

### BACKGROUND

### Field

The present disclosure relates to continuous glucose monitoring (CGM). More specifically, it relates to CGM sensors.

### Description of the Related Art

Continuous glucose monitoring (CGM) sensors based on electrochemical methods may be capable of detecting glucose by indirect measurement of a molecule that is generated during an enzymatic reaction. The enzymatic product may be, as examples, hydrogen peroxide, an artificial mediator, and/or the reduced enzyme co-factor itself. The common aspect for all these measurements is the application of an electrochemical potential sufficient to oxidize these molecules to generate a current.

### SUMMARY

The devices, systems, and methods of the present disclosure each have several innovative aspects, no single one of which is solely responsible for its desirable attributes. Without limiting the scope of the present disclosure, its more prominent features will be discussed herein.

Aspects of the present disclosure relate to an electrochemical probe. The electrochemical probe can include an electrode, an enzyme layer; and an interference zapping layer.

In some aspects, the probe can additionally include a glucose limiting layer. In some aspects, the electrode can be capable of measuring glucose concentration. In some aspects, the enzyme layer can be capable of converting glucose to hydrogen peroxide and gluconic acid. In some aspects, the enzyme layer includes a glucose oxidase. In some aspects, the probe includes a blocking layer. In some aspects, the blocking layer can include a size-based filter or an electrostatic repulsion filter. In some aspects, the probe may include a voltage source that can set the electrode to an applied potential of +0.1 to +1 V. In some aspects, the probe may include a voltage source that can set the electrode to an applied potential of +0.6 to +0.7 V. In some aspects, the probe may include a voltage source that can set the interference zapping layer to an applied voltage of +0.3 to +1 V. In some aspects, the probe may include a voltage source that can set the interference zapping layer to an applied voltage of +0.7 to +0.8 V. In some aspects, the probe may include a first voltage source that can set the electrode to a first applied potential, and a second voltage source that can set the interference zapping layer to a second applied potential, where the second applied potential may be equal to or higher than the first applied potential. In some aspects, the interference zapping layer may include a hydrogel. In some aspects, the interference zapping layer may include a microwire. In some aspects, the interference zapping layer may include a microwire network. In some aspects, the interference zapping layer may include a nanowire. In some aspects, the interference zapping layer may include a nanowire network. In some aspects, the interference layer may include cellulose acetate crosslinked to citric acid. In some aspects, a continuous glucose monitor may include an electrochemical biosensor probe in accordance with the present disclosure.

Aspects of the present disclosure relate to a probe. The probe may include a first electrode in contact with a first enzyme layer; a second electrode; and a first interference zapping layer exterior to the first electrode and second electrode. In some aspects, the probe may include an insulating substrate positioned under the first electrode and second electrode. In some aspects, the probe may include a third electrode in contact with a second enzyme layer. In some aspects, the probe may include a second enzyme layer in contact with the second electrode; and a first polymeric layer exterior to the first interference zapping layer. In some aspects, the probe may include a second interference zapping layer exterior to the first polymeric layer; and a second polymeric layer exterior to the second interference zapping layer. In some aspects, the probe may include the first enzyme layer including a glucose oxidase; and the second enzyme layer including a catalase.

Aspects of the present disclosure relate to a method of using a probe. The method may include applying a first potential to an interference layer, the first potential sufficient to oxidize at least one electrochemical interferent and a molecule of interest; measuring a first background current of a first electrode; measuring a second background current of a second electrode; applying a second potential to the interference layer, the second potential sufficient to oxidize at least one electrochemical interferent but not oxidize the molecule of interest; measuring a first current of the first electrode; measuring a second current of the second electrode; determining an estimate of the concentration of the molecule of interest based, at least in part, on measurements of the first background current, the second background current, the first current, and the second current. In some aspects, the molecule of interest includes glucose. In some aspects, the first potential may be within +0.5 to +1.5 V. In some aspects, the first potential may be within +0.6 to +1.1 V. In some aspects, the first potential may be within +0.1 to +0.9 V. In some aspects, the second potential may be within +0.3 to +1.1V. In some aspects, the second potential may be within +0.4 to 0.7 V. In some aspects, the second potential may be within +0.1 to +0.9 V.

Aspects of the present disclosure relate to a method of using an electrochemical probe including an interference zapping layer and a working electrode. The method may include applying a first plurality of potentials to the interference zapping layer; applying a second plurality of potentials to the working electrode; measuring a plurality of currents of the working electrode, each of the plurality of currents measured while the interference zapping layer may be set to one of the first plurality of potentials and while the working electrode may be set to one of the second plurality of potentials; determining, with a hardware processor, an estimate of a concentration of an analyte based, at least in part, on the measured plurality of currents.

In some aspects, the method may include determining an estimate of the concentration of a plurality of analytes based, at least in part, on the measured plurality of currents. In some aspects, applying the first plurality of potentials to the interference layer includes sequentially applying the first plurality of potentials. In some aspects, applying the second plurality of potentials to the working electrode includes sequentially applying the second plurality of potentials. In some aspects, each of the plurality of currents may be measured at a different combination of one of the first plurality of potentials and one of the second plurality of potentials. In some aspects, the first plurality of potentials may be a series having a step of Δ±0.1 V between each of the first plurality of potentials. In some aspects, the second plurality of potentials may be a series having a step of Δ±0.1 V between each of the first plurality of potentials. In some aspects, while the interference layer may be held at one of the first plurality of potentials, the potential applied to the working electrode steps through the second plurality of potentials. In some aspects, while the working electrode may be held at one of the second plurality of potentials, the potential applied to the interference electrode steps through the first plurality of potentials. In some aspects, the method does not include a calibration step.

Aspects of the present disclosure relate to a method of using an electrochemical probe comprising an interference zapping layer and a working electrode, the method including: applying a first plurality of potentials to the interference zapping layer; applying a second plurality of potentials to the working electrode; measuring a first plurality of currents of the working electrode, each of the first plurality of currents measured while the interference zapping layer is set to one of the first plurality of potentials and while the working electrode is set to one of the second plurality of potentials; determining, with a hardware processor, a third plurality of potentials, the third plurality of potentials comprising at least some of the first plurality of potentials; determining, with a hardware processor, a fourth plurality of potentials, the fourth plurality of potentials comprising at least some of the first plurality of potentials; and measuring a second plurality of currents of the working electrode, each of the second plurality of currents measured while the interference zapping layer is set to one of the third plurality of potentials and while the working electrode is set to one of the fourth plurality of potentials.

In some aspects, the third plurality of potentials consists of at least one of the first plurality of potentials, and the fourth plurality of potentials consists of at least one of the second plurality of potentials. In some aspects, determining the third plurality of potentials may be based at least in part on the first plurality of currents indicative of an analyte. In some aspects, determining the fourth plurality of potentials may be based at least in part on the first plurality of currents indicative of an analyte. In some aspects, the second plurality of currents includes fewer currents than the first plurality of currents. In some aspects, measuring the second plurality of currents may be quicker than measuring the first plurality of currents. In some aspects, measuring the second plurality of currents may require less power than measuring the first plurality of currents.

Aspects of the present disclosure relate to a method of using an electrochemical probe comprising an interference zapping layer and a working electrode, the method including: applying a first plurality of potentials to the interference zapping layer; applying a second plurality of potentials to the working electrode; measuring a plurality of currents of the working electrode, each of the plurality of currents measured while the interference zapping layer is set to one of the first plurality of potentials and while the working electrode is set to one of the second plurality of potentials; constructing, with a hardware processor, a data structure; and determining, with the hardware processor and based at least in part on the data structure, an estimate of an analyte concentration.

In some aspects, the method includes displaying the analyte concentration on a display. In some aspects, the data structure is an array. In some aspects, the data structure is a heat map. In some aspects, the data structure is a three-dimensional plot. In some aspects, determining an estimate of an analyte concentration comprises comparing the data structure to a control data structure. In some further aspects, the control data structure includes current measurements of a control subject. In some further aspects, the control data structure comprises current measurements of a control fluid. In some further aspects, the control data structure comprises a previous measurement of a previous plurality of currents of the patient. In some aspects, the method includes identifying, using the hardware processor, physiological changes based at least in part on differences between the data structure and the previous measurement. In some aspects, the method includes positioning the interference zapping layer and working electrode within interstitial fluid of a patient.

### BRIEF DESCRIPTION OF THE DRAWINGS

The above-mentioned aspects, as well as other features, aspects, and advantages of embodiments of the present disclosure will now be described in connection with various implementations and/or embodiments, with reference to the accompanying drawings. The illustrated implementations are merely examples and are not intended to be limiting. Throughout the drawings, similar symbols typically identify similar components, unless context dictates otherwise.

FIG. 1 illustrates a reaction diagram of the conversion of glucose to gluconic acid and hydrogen peroxide.

FIG. 2 illustrates an example sensor having a blocking layer.

FIGS. 3A-3C illustrate example sensors each having an interference zapping layer.

FIGS. 4A-4C illustrate the independent circuits of the electrodes and interference zapping layers of the sensors of FIGS. 3A-3C.

FIGS. 5A-5C illustrate various multi-electrode sensors.

FIG. 6 illustrates an example method using the sensor of FIG. 5A to correct for background current.

FIG. 7 illustrates a scheme for a measurement mode where a plurality of discrete measurements are generated, each discrete measurement corresponding to a particular combination of working electrode potential and interference zapping layer potential.

FIG. 8A-8D illustrates processes for measuring a plurality of currents corresponding to a range of working electrode potentials and a range of interference zapping layer potentials.

FIG. 9 is an example heatmap of measured currents.

FIG. 10 illustrates a process for selectively measuring a subset of currents of the scheme of FIG. 7.

FIG. 11A is a continuous plot of current as a function of working electrode potential for various concentrations of glucose.

FIG. 11B is a continuous plot of current as a function of working electrode potential for various concentrations of acetaminophen.

FIGS. 12A and 12B schematically illustrate example sensors capable of rapid hydration.

### DETAILED DESCRIPTION

Aspects of the present disclosure provide devices, systems, and methods capable of optimizing in vivo electrochemical measurement of a molecule of interest and/or analyte, for example glucose. Such aspects may include an interference zapping layer, to which an electric potential may be applied to prevent or minimize various interfering molecules reaching a working electrode. Aspects of the present disclosure may additionally or alternatively include a sensor including three electrodes. These electrodes may dynamically measure background current during sensing in order to determine current due to electrochemical interferents and current due to the molecule of interest.

### Electrochemical Interferents and Background Current

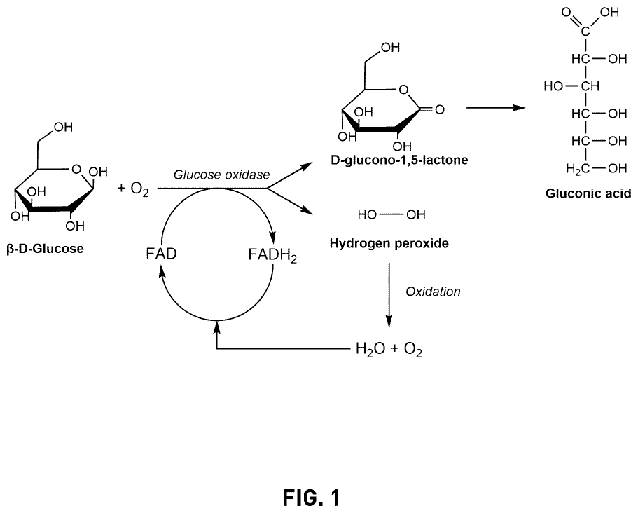

In certain instances where an electrochemical sensor can sense an analyte concentration, such as, for example, glucose concentration, enzymatic conversion may generate hydrogen peroxide from glucose at electrodes, for example platinum electrodes, so that the concentration of hydrogen peroxide corresponds to glucose concentration. FIG. 1 illustrates the conversion of glucose to gluconic acid. The enzymatic conversion of glucose to gluconic acid and hydrogen peroxide may be accomplished by, for example, a glucose oxidase.

To enable detection of hydrogen peroxide, the electrode may need an applied potential, for example +0.6-0.7 V vs. Ag/AgCl, at the working electrode. In certain instances, the electrochemical potential of a sensor electrode can be in the range of 0.0 to +0.7 V vs. Ag/AgCl. In certain conditions, the current generated by oxidation of hydrogen peroxide, which generates H₂O and O₂, at an applied potential may directly correlate to glucose concentration.

However, there are other molecules within body fluids that contact the sensor that can undergo electrochemical reactions at the electrode surface in these potential range. If these other molecules do undergo electrochemical reactions, they may produce background currents that may be read by the sensor electrode. These background current-causing molecules are referred to as “electrochemical interferents” or “interferents” herein. While some electrochemical interferents can be naturally present in body fluids and/or generated by cells and released in body fluids (e.g., ascorbic acid, uric acid, reactive oxygen species, etc.), some are introduced by ingestion (e.g., acetaminophen, ibuprofen, ascorbic acid). Variation of the concentration of electrochemical interferents in body fluids, either due to normal physiological processes or by external factors including ingestions, can lead to variation in the background signals during sensing. Without any means to eliminate the electrochemical interferents, the resultant current measured at the working electrode may be a convolution of all the electro-active molecules, including, for example, hydrogen peroxide generated by the glucose oxidase enzyme. Variation in background signals can thus lead to erroneous estimation of target molecule concentration, for example glucose concentration, in the body fluid. For analyte sensing, for example for glucose sensing, it may therefore be desirable to minimize oxidation of molecules other than hydrogen peroxide at the electrode.

One method to overcome this problem may be to modify the surface of the sensor to block the interferents from reacting at the electrode surface. For example, existing strategies to eliminate interfering molecules include either using the property of electrostatic repulsion of charged molecules, for example ascorbic acid and/or uric acid, etc., and/or size-based filtering for neutrally charged molecules, for example acetaminophen, etc. Such blocking may be passive. However, these strategies may have shortcomings. For example, a size-based elimination strategy may not block the endogenous hydrogen peroxide or other reactive oxygen species (ROS) that are products of normal physiological function. Further, during continuous operation, the blocking layer may disintegrate, leading to a higher background current.

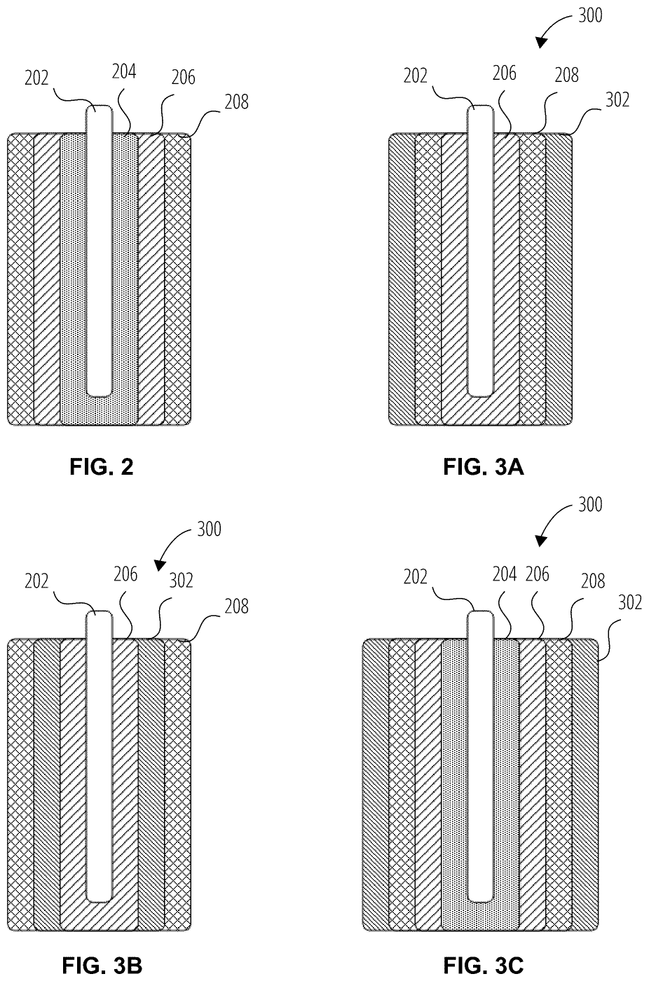

FIG. 2 diagrams an example construction of a sensor 300 illustrating an approach to eliminate interfering molecules using a blocking layer. The example electrochemical sensor 300 includes one or more working electrode 202, a blocking layer 204, an enzyme layer 206, and an analyte limiting layer 208. The working electrode 202 may include a metal material, for example platinum. The blocking layer 204 may include a porous material with pores sized to allow passage of a target molecule but small enough to bar passage of molecules larger than the molecule of interest. Additionally or alternatively, the blocking layer 204 may incorporate a static charge which allows passage of a target molecule but blocks molecules having a dissimilar charge to the molecule of interest. The enzyme layer 206 may include an enzyme, for example glucose oxidase, for converting a molecule of interest, for example glucose, into a target molecule, for example hydrogen peroxide.

When operating, the electrode may be maintained at an appropriate applied potential, for example a potential within the range of +0.6-0.7 V. At this potential, hydrogen peroxide may be generated by the enzyme layer 206 as a byproduct of conversion of glucose to gluconic acid. At the working electrode 202, hydrogen peroxide may be oxidized to generate a current that may be, at least in part, proportional to glucose concentration.

To eliminate the interfering molecules, the blocking layer 204 may act to filter molecules based on size exclusion or by leveraging molecular properties such as static charge on the interfering molecules at physiological pH. The order of blocking layer 204 and enzyme layer 206 can be interchanged depending on the application (e.g. the enzyme layer 206 could be proximate to the working electrode 202 and the blocking layer 204 could be layered on the exterior of the enzyme layer 206). The third layer, the analyte limiting layer 208, may proportionally equalize the molar concentration of the analyte and a reactant involved in an enzymatic reaction to create a target molecule. In aspects where the analyte limiting layer 208 is a glucose limiting layer (e.g., for a glucose sensor), the analyte limiting layer 208 may proportionally equalize the molar concentration of glucose and oxygen at the enzyme layer to shift the enzyme reaction to be glucose-dependent. When the electrode operates in typical physiological conditions, there may be 100- to 1000-fold more glucose than oxygen molecules. It may be desirable that the analyte limiting layer 208 may be fabricated to enclose the enzyme layer 206 so that it may effectively proportionally equalize the molar concentration of glucose and oxygen at the enzyme layer 206.

Another approach may be to use an additional electrode, not having an enzyme layer, to measure the current from the background interferents. However, this strategy often fails to estimate the accurate glucose concentration by simple subtraction of interferent current (measured by the electrode without an enzyme layer) from the signal current (measured by the electrode including an enzyme layer). This failure stems from an assumption that the background currents of both electrodes are the same value, which may be not be the case. The true background of any two electrodes can vary for several reasons, including minor differences in their surface roughness, their history of chemical or biological modification, time-evolved adsorption of species, changes undergone during exposure to biological fluid during sensing, etc. These issues may cause relatively large errors when the current signals are small relative to the background currents, as may be the case for CGM sensors.

### Overview

Herein are disclosed sensors and electrochemical methods to address the problems discussed above. Sensors in accordance may be inserted into a patient's body. A sensor in accordance with the present disclosure may be inserted into the patient's body such that it contacts the patient's bodily fluid. For example, a sensor in accordance with the present disclosure may be inserted into the patient's body such that it at least partially comes in contacts the patient's interstitial fluid. For example, a sensor in accordance with the present disclosure may be inserted into the patient's body such that it contacts the patient's blood.

In one aspect, the present disclosure provides for inclusion of a novel interference zapping layer around and/or over the working electrode of a CGM sensor. The interference zapping layer exploits the electroactivity of electrochemical interferents to filter out interfering molecules before they reach the enzyme layer and/or working electrode. The interference zapping layer may thereby eliminate electrochemical interferents.

In another aspect, the present disclosure provides for a sensor including three electrodes to dynamically measure the background current of each electrode during a sensing duration, measure the contribution due to electrochemical interferents, and use these measurements to accurately estimate the glucose concentration in the biological fluid of interest.

In another aspect, the present disclosure provides for optional inclusion of a wetting layer in a sensor in accordance with the present disclosure. Such a wetting layer may accelerate sensor hydration, thereby shortening the “warm-up” period after insertion of the sensor into a patient.

In yet another aspect, the present disclosure provides for a method, referred to herein as a “measurement mode,” for measuring concentration of one or more analytes using a prove having an interference zapping layer in accordance with the present disclosure.

Though this disclosure discusses the conversion of enzymatic glucose to generate hydrogen peroxide which may subsequently be detected by an electrode, one having skill in the art would recognize that the concepts disclosed herein may apply to the sensing of other molecules of interest. For instance, this disclosure may be generally applicable to any electrochemical sensor that (1) operates at an applied potential, (2) includes metal in its working electrode(s) and/or (3) includes one or more chemistry layers for in the sensor.

### Interference Zapping Layer

In one aspect, the present disclosure provides for usage of an interference zapping layer which exploits the oxidizability of various electrochemical interferents to block them from reaching, for example by diffusion, to a working electrode.

There are many interfering molecules in normal human physiology that are electrochemically oxidizable at various voltages across the range 0 to +1 V. For a given electro-active molecule, maintaining a potential slightly higher than its peak potential may oxidize the electro-active molecule. Peak potential may be determined by voltammetry, as described in Naghian, E. et al., Carbon paste electrodes modified with SnO₂/CuS, SnO₂/SnS and Cu@SnO₂/SnS nanocomposites as voltammetric sensors for paracetamol and hydroquinone, Microchimica Acta 185, 406 (2018). Local peaks for the eight interfering molecules examined by Naghian et al using voltammetry measured fall approximately within the 0 to +1 V range.

The interference zapping layer may allow for tunable control over which molecular species reach the working electrode. For example, the potential of an interference zapping layer may be adjustable, even during use of the probe, in contrast to a blocking layer, which can only passively block interferents based on size and/or charge and cannot be adjusted during use.

An interference zapping layer according to the present disclosure can exploit the oxidizability of electrochemical interferents. For example, the interference zapping layer may oxidize all, substantially all, and/or many molecules that are electrochemically active. The interference zapping layer's efficiency may depend on the applied voltage and the density of the interference zapping layer's wire network. As described in accordance with the present disclosure, both wire network density and voltage can be controlled. If the applied potential at the interference zapping layer is at or above the working potential of the analyte sensing electrode and the wire network is sufficiently dense, all, substantially all, and/or many electrochemical interferents may be blocked before reaching the sensing electrode. The interference zapping layer may also efficiently block unknown, poorly understood, and/or novel interfering molecules.

As an illustrative example of blocking by the interference layer, hydrogen peroxide may be endogenously produced as a byproduct of metabolism and/or inflammation. Endogenous hydrogen peroxide is not substantially blocked by either size-based or charge-based blocking layer approaches. However, an interference zapping layer constructed in accordance with the present disclosure can oxidize, and thereby substantially block, the hydrogen peroxide molecules that are either outwardly leaving the underlying enzymatic layer and/or oxidize endogenously generated hydrogen peroxide molecules diffusing inward to the interference zapping layer. The rate at which the interference zapping layer blocks hydrogen peroxide may depend on the geometry of it and other layers, the wire network density, and/or other factors.

In some examples, the zapping layer may block interfering molecules for a glucose sensing electrode that operates at lower potentials, for example +0.1 to +0.4 V vs. Ag/AgCl. In these examples, the applied potential of the interference zapping layer may be held at +0.45 V vs. Ag/AgCl or some other appropriate voltage.

### Sensors Including an Interference Zapping Layer

In some examples, a sensor 300 includes at least three chemical/biochemical layers that each may perform specific functions. For example, the three functions may include: biochemical conversion (performed by an enzyme), balancing reactants (performed by an analyte limiting layer), and filtration (by electrochemical oxidation as performed by an interference zapping layer, disclosed herein).

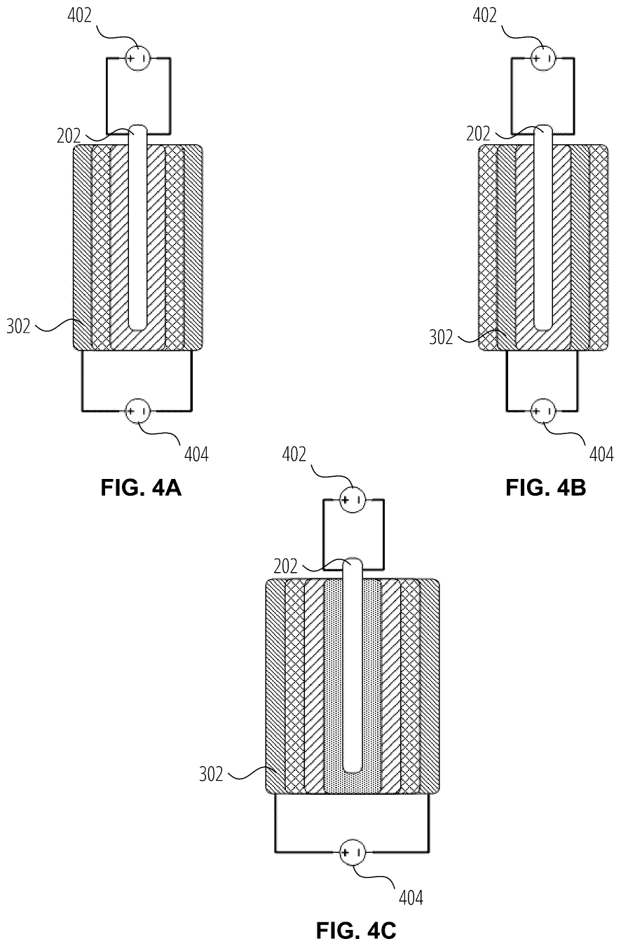

FIG. 3A, FIG. 3B, and FIG. 3C diagram example layering schemes for sensor 300. The example sensor 300 depicted in FIG. 3A includes a sensing electrode 202 (also referred to as a working electrode), an enzyme layer 206, a analyte limiting layer 208, and an interference zapping layer 302. The example sensor 300 depicted in FIG. 3B includes the same types of layers, but the order of the analyte limiting layer 208 and the interference zapping layer 302 may be switched. Unlike in FIG. 3A, the analyte limiting layer 208 of FIG. 3B may be exterior to the analyte limiting layer 208. In some aspects, the interference zapping layer 302 may be the outermost layer of the sensor 300, as illustrated in FIG. 3A. In some aspects, the interference zapping layer 302 may be between the analyte limiting layer 208 and the enzyme layer 206, as illustrated in FIG. 3B.

The example sensor 300 depicted in FIG. 3C includes, from innermost to outermost, a sensing electrode 202, a blocking layer 204, an enzyme layer 206, an analyte limiting layer 208, and an interference zapping layer 302. Other example sensors including the layers depicted in FIG. 3C may reorder the layers as set forth in this disclosure. In some aspects, the enzyme layer 206 may include polyurethane, polyethylene glycol diglycidyle ether, and/or polyethylene diamine. In some aspects, the enzyme layer 206 may include an enzyme capable of generating a target molecule from an analyte of interest. In some aspects, the enzyme of the enzyme layer 206 may be a glucose oxidase. In some aspects, the analyte limiting layer 208 may include a polyurethane.

The interference zapping layer 302 may be conductive, at least in part. As discussed in further detail herein, the interference zapping layer 302 may include a conductive wire network, for example a microwire network and/or a nanowire network. A microwire network may include wires having diameter of approximately 1 μm to 1 mm. A nanowire network may include wires having diameter of approximately 1 nm to 1 μm. In the examples discussed above, electrochemical interferents can be oxidized, and thereby blocked from a sensing electrode 202 by applying a voltage to an interference zapping layer 302 layered exterior to the enzyme layer 206. An interference zapping layer 302 may be electrically conductive to facilitate application of a potential. In some implementations, the sensing electrode 202 may be held at a potential of between approximately 0.0 and +2.0 V, a potential of between approximately 0.0 and +1.75 V, a potential of between approximately 0.0 and +1.5 V, a potential of between approximately 0.0 and +1.25 V, a potential of between approximately 0.0 and +1.0 V, a potential of between approximately 0.0 and +0.9 V, a potential of between approximately 0.0 and +0.8 V, a potential of between approximately 0.0 and +0.7 V, a potential of between approximately 0.0 and +0.6 V, a potential of between approximately 0.0 and +0.5 V, a potential of between approximately 0.0 and +0.4 V, a potential of between approximately +0.1 and +1.0 V, a potential of between approximately +0.1 to +0.4 V, a potential of between approximately +0.6 to +0.7 V, or any value or range within or bounded by any of these ranges or values, although values outside these values or ranges can be used in some cases.

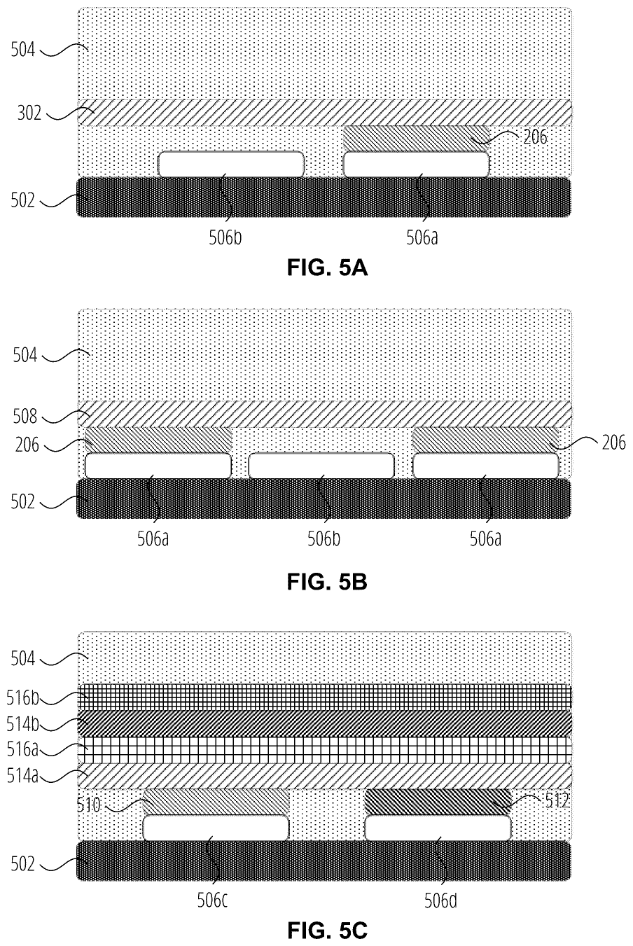

In certain implementations, the interference zapping layer 302 may be held at an applied potential similar to that of the sensing electrode 202, for example +0.6 to +0.7 V, or slightly higher, for example +0.7-0.8 V. The interference zapping layer 302 may be in an independent electrochemical circuit to the sensing electrode 202, as illustrated by FIG. 4A, FIG. 4B, and FIG. 4C. The example sensor of FIG. 4A diagrams circuits of the sensor of FIG. 3A. The example sensor of FIG. 4B diagrams circuits of the sensor of FIG. 3B. The example sensor of FIG. 4C diagrams circuits of the sensor of FIG. 3C. Applied electrode potential 402 and applied interference zapping layer potential 404 may be independent. In some implementations, the interference zapping layer 302 may be held at a potential of between approximately 0.0 and +2.0 V, a potential of between approximately 0.0 and +1.75 V, a potential of between approximately 0.0 and +1.5 V, a potential of between approximately and +1.25 V, a potential of between approximately 0.0 and +1.0 V, a potential of between approximately 0.0 and +0.9 V, a potential of between approximately 0.0 and +0.8 V, a potential of between approximately 0.0 and +0.7 V, a potential of between approximately 0.0 and +0.6 V, a potential of between approximately 0.0 and +0.5 V, a potential of between approximately and +0.4 V, a potential of between approximately +0.1 and +1.0 V, a potential of between approximately +0.1 to +0.4 V, a potential of between approximately +0.2 to +0.5 V, a potential of between approximately +0.3 to +0.6 V, a potential of between approximately +0.6 to +0.7 V, a potential of between approximately +0.7 to +0.8 V, or any value or range within or bounded by any of these ranges or values, although values outside these values or ranges can be used in some cases.

In some aspects, the analyte limiting layer 208 may be 1, 2, 3, 4, 5, 6, 7, 8, 9, 10, 11, 12, 13, 14, or 15 μm thick, or within a range defined by any of the preceding values. In some aspects, the thickness of the analyte limiting layer 208 may be from 2 to 10 μm. Increasing the thickness of the analyte limiting layer 208 may result in more glucose being blocked. Increasing the thickness of the analyte limiting layer 208 may result in lower current measured by the electrode 202.

In some aspects, the enzyme layer 206 may be 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 μm thick, or within a range defined by any of the preceding values. In some aspects, the thickness of the enzyme layer 206 may be from 1 to 5 μm.

In some aspects, the interference blocking layer 302 may be 0.5, 1.0, 1.5, 2.0, 2.5, 3.0, 3.5, 4.0, 4.5, 5.0, 5.5, 6.0, 6.5, 7.0, or 7.5 μm thick, or within a range defined by any of the preceding values. In some aspects, the thickness of the interference blocking layer 302 may be from 2 to 5 μm.

### Composition and Fabrication of an Interference Zapping Layer

An interference zapping layer 302 may be fabricated in any one of many suitable ways. The interference zapping layer 302 may include a conductive element, for example a wire such as a microwire or a nanowire. The interference zapping layer 302 may be an electrode that may allow the diffusion of molecules through it. In one example, conductive wire may be assembled exterior to the sensing electrode 202 either in parallel to the length of the sensing electrode 202, or in a coiled fashion around the sensing electrode 202. The wire may be arranged such that there may be adequate separation so diffusion of the analyte to the underlying electrode is not impeded. The wire may be arranged as a network, for example. As another example, the zapping layer may be constructed by embedding a wire network, for example a platinum nanowire network, in a polymer matrix. This method may be desirable because it may be suitable for large-scale manufacturing. The wire, wire network, and/or interference zapping layer may be synthesized according to procedures described in Shi, Q. et al., Mesoporous Pt Nanotubes as a Novel Sensing Platform for Sensitive Detection of Intracellular Hydrogen Peroxide, Acs Appl Mater Inter 7, 24288-95 (2015) and Shi, Q. et al., Kinetically controlled synthesis of AuPt bi-metallic aerogels and their enhanced electrocatalytic performances, J Mater Chem A 5, 19626-31 (2017), incorporated herein by reference. A wire network may be formulated into a polymer matrix, for example a hydrogel or any other suitable material, such that the wire network may be deposited as a self-contained interference zapping layer 302. The wire may be arranged within the interference zapping layer 302 such that there may be adequate separation to allow diffusion of the molecule of interest to the underlying sensing electrode 202. In another exemplary fabrication method, a conductive wire network can be coated with a colloidal suspension and subsequently coated with a polymer matrix to preserve its integrity.

In the example sensors shown in FIG. 3A, FIG. 3B, and FIG. 3C, the conducting nanomaterial network within the interference zapping layer 302, held at a given applied potential, may oxidize all, substantially all, a majority of, or a fraction of the electrochemical interferents that are electroactive below the chosen applied potential that are within and/or near the interference zapping layer 302, thereby preventing and/or inhibiting at least some electrochemical interferents from reaching the working electrode 202 itself. In some instance, a molecule of interest may not be oxidizable at certain applied potentials. For example, glucose is not oxidizable at certain applied potentials, for example +0.7-0.8V. At such potentials, at least some glucose may pass through the interference zapping layer even while at least some electrochemical interferents cannot.

The density of the wire network, for example the nanowire network and/or microwire network, within the interference zapping layer 302 may determine the efficacy of excluding electrochemical interferents. Too low a density may not effectively exclude electrochemical interferents. However, too dense a network may impede the diffusion of all molecules including molecules of interest, for example glucose. Wire density can be controlled by, for example, increasing the concentration of wires during fabrication of the interference zapping layer 302. Additionally or alternatively, wire density can be controlled by adding multiple interference zapping layers 302 to a single sensing electrode 202. Additionally or alternatively, network density can be controlled by the choice of the polymer matrix included in the interference zapping layer 302. There are many off-the-shelf polymer matrix materials which may be biocompatible and potentially suitable for inclusion in an interference zapping layer 302. In some aspects, the interference zapping layer 302 includes cellulose acetate.

In some aspects, the interference zapping layer 302 may optionally include one or more crosslinkers. The interference zapping layer 302 may be more effective at blocking electrochemical interferents as its thickness increases. Increasing the thickness of the interference zapping layer 302 may cause the interference zapping layer 302 to be more susceptible to cracking, however, which can decrease blocking efficiency. In aspects where the interference zapping layer 302 includes cellulose acetate, the cellulose acetate may dissolve when exposed to organic solvents used to form other layers of the sensor. This may cause difficulty in controlling uniformity in the thickness of the interference zapping layer 302. In some aspects, the interference zapping layer 302 may include crosslinkers between cellulose acetate and citric acid. In some aspects, a crosslinker between cellulose acetate and citric acid can be introduced using a one-step method involving a solid-state reaction. In some aspects, the crosslinking reaction may proceed according to Scheme 1:

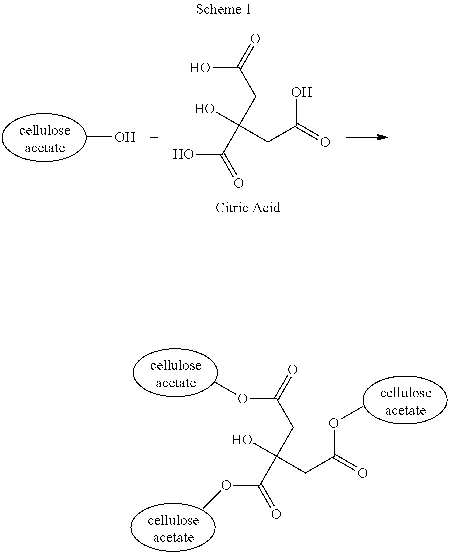

The crosslinking reaction of Scheme 1 may proceed at 130° C. for about 40 minutes. The crosslinking reaction of Scheme 1 may be used for interference zapping layers of thickness of 2-5 μm. An interference zapping layer subjected to a crosslinking reaction in accordance with Scheme 1 may be more resistant to cracking and/or more resistant to dissolving when exposed to organic solvents than an interference zapping layer without crosslinking.

### Multi-Electrode Sensors

In another aspect, the present disclosure provides for the use of multi-electrode sensors to correct for background current.

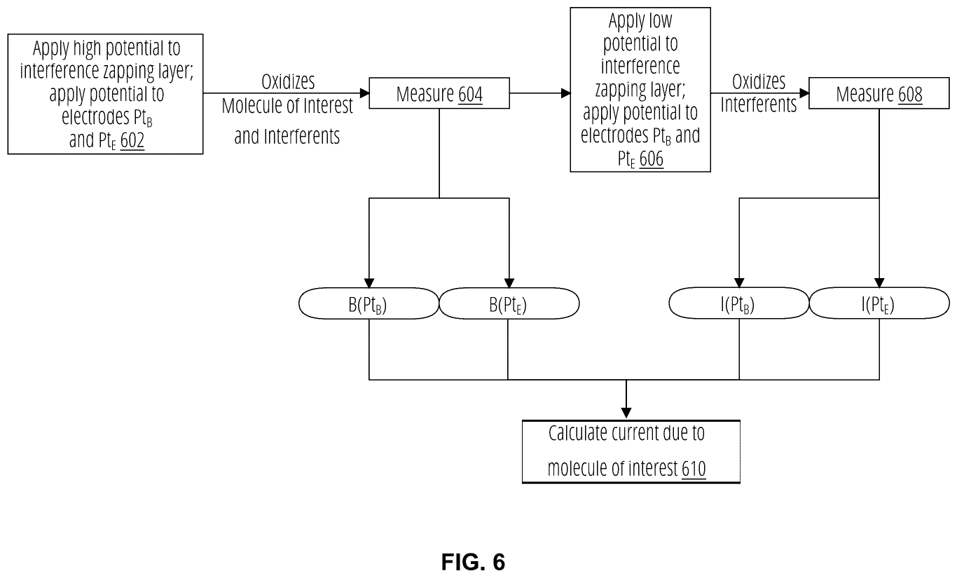

FIG. 5A illustrates an example of an example multi-electrode sensor. The sensor includes electrodes for the proposed method to estimate and eliminate the background currents due to interferents and estimate the change in enzyme activity with time. Two electrodes, 506a and 506b, are situated on an insulating substrate 502 and one porous electrode, interference zapping layer 302, may be positioned directly above the two electrodes 506a and 506b. All three electrodes 302, 506a, and 506b can be independently electrochemically controlled. The porosity of the top interference zapping layer 302 allows for a molecule of interest, for example glucose, in the body fluid 504 to diffuse through the interference zapping layer 302 to the sensing layer. One of the electrodes, 506a, may be associated with an enzyme layer 206. The enzyme layer 206 may convert the molecule of interest, for example glucose, into a target molecule, for example hydrogen peroxide, that may be sensed by the electrode 506a. In an example CGM sensor, the enzyme layer 206 may oxidize glucose in the presence of oxygen to release hydrogen peroxide. The target molecule can be detected at the underlying electrode 506a surface to generate a sensing signal. Another electrode 506b may be situated next to the electrode 506a. The electrode 506b may not have an associated enzyme layer. Hence, in an example CGM sensor, electrode 506b may provide a signal proportional to the currents due to oxidation of all, substantially all, or a portion of the electrochemical interferents in the body fluid within the sensor, except for the molecule of interest. To ensure that the signal sensed by electrode 506b does not correspond to oxidation of the molecule of interest, it may be desirable to set electrode 506b to a potential at which the molecule of interest cannot be electrochemically oxidized. For aspects where glucose may be the molecule of interest, it may be desirable to set electrode 506b to +0.6 to +0.7 V, for example. The third porous electrode, interference zapping layer 302 placed on top of the two electrodes 506a and 506b, if set to an appropriate potential, for example +0.6 to +0.7 vs. Ag/AgCl, can oxidize and thereby eliminate electrochemical interferents as disclosed herein. Additionally or alternatively, interference zapping layer 302 can oxidize the molecule of interest and electrochemical interferents if set to a relatively high potential. For aspects where glucose may be the molecule of interest, setting the interference zapping layer 302 higher than +0.8 V vs. Ag/AgCl may oxidize both glucose and electrochemical interferents.

FIG. 5B illustrates an example multi-electrode sensor. In addition to the elements discussed with reference to FIG. 5A, the sensor of FIB. 5B includes two electrodes 506a and associated enzyme layers 206. Inclusion of two electrodes 506a with enzyme layers 206 enables the reading of an average signal over the length of the sensor. For example, the two electrodes 506a may be able to measure an average signal due to glucose. In certain examples, more electrodes 506a and enzyme layers 206 and/or electrodes 506b may be included in the sensor. Inclusion of multiple electrodes may reduce or eliminate the error in estimating the concentration of a molecule of interest, for example glucose, that can arise from a concentration gradient along the sensor's length.

FIG. 5C illustrates an example multi-electrode sensor. The example sensor may be used to sense a molecule of interest, for example glucose. The sensor includes insulating substrate 502, electrodes 506c and 506d, a catalase layer 510, an enzyme layer 512, a first interference zapping layer 514a, a first polymeric layer 516a, a second interference zapping layer 514b, and a second polymeric layer 516b. In some aspects where the molecule of interest is glucose, the enzyme layer 512 may include a glucose oxidase. The sensor may estimate and eliminate the background currents due to electrochemical interferents and estimate the change in enzyme activity of the enzyme layer 512 over time. The two electrodes 506c and 506d may be situated on an insulating substrate 502 and the two porous electrodes, first interference zapping layer 514a and second interference zapping layer 514b, may be situated above the electrodes 506c and 506d. The two porous electrodes, first interference zapping layer 514a and second interference zapping layer 514b may be separated by polymeric layers, first polymeric layer 516a and second polymeric layer 516b, positioned above the two electrodes 506c and 506d. All four electrodes 506c, 506d, 514a, and 514b can be independently electrically controlled. The porosity of the interference zapping electrodes 514a and 514b may allow the molecule of interest to diffuse through to the enzyme layer 512. Composition of the first interference zapping layer 514a and the second interference zapping layer 514b may be in accordance with the composition for interference zapping layers generally, as described herein.

The electrode 506d may be coated with the enzyme layer 512, in which the molecule of interest, for example glucose, can undergo oxidation in presence of oxygen to release a target molecule, for example hydrogen peroxide. The target molecule can be detected at the underlying electrode 506d surface, where the hydrogen peroxide may generate a current corresponding to the concentration of the molecule of interest. The electrode 506c, which may be positioned next to the electrode 506d, may be coated with the catalase layer 510. The catalase layer 510 can prevent target molecules reaching the electrode 506c from the enzyme layer 512. It may be desirable to set the potential of electrode 506c to a potential at which the molecule of interest cannot be electrochemically oxidized such that the signal sensed by electrode 506c does not include, or minimizes signal of the electrode 506c due to the molecule of interest. In aspects where glucose is the molecule of interest, the potential of electrode 506c may be set, for example, to +0.6 to +0.7 V, to ensure that the signal sensed by electrode 506c is not due to glucose concentration and/or minimally due to glucose concentration. The electrode 506c can thus provide a current signal proportional to all the electrochemical interferents in the body fluid except for the molecule of interest, for example glucose. The second interference zapping layer 514b situated on top of the two electrodes 506c and 506d, when set to an appropriate potential, for example at a potential of approximately +0.6 V vs. Ag/AgCl, can oxidize interferents. The first interference zapping layer 514a, when set to an appropriate potential, for example of approximately +0.6 V vs. Ag/AgCl, can oxidize molecules of interest, for example glucose and glucose-like molecules. Advantageously, the potentials applied at the interference zapping layers may be relatively mild and can prevent and/or reduce degradation of the polymeric layers in contact with the interference zapping layers.

### Methods of Calibrating for Background Interferents In Vivo Using Multi-Electrode Sensors

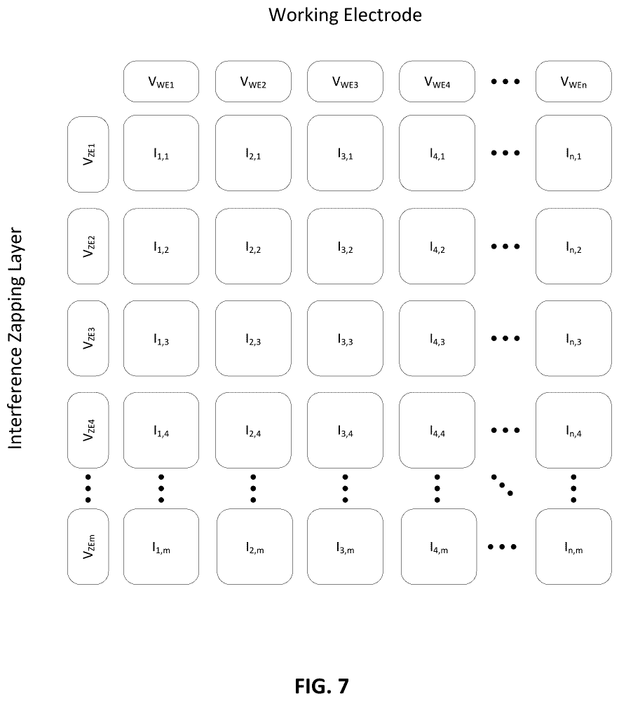

FIG. 6 illustrates an example method of calibrating for background interferents in vivo. The method of FIG. 6 may apply to a sensor, for example the sensor diagrammed in FIG. 5A. Within FIG. 6, the electrode with enzyme layer 506a is referred to as Pt_{E}, while the electrode without enzyme layer 506b is referred to as Pt_{B}. Though FIG. 6 discloses use of certain potential values, it is to be understood that any potential for oxidizing and/or eliminating certain molecules of interest, target molecules, and/or electrochemical interferents may be used in the various steps disclosed. Additionally, it is to be understood that glucose is an example molecule of interest, but one having skill in the art would recognize that the method shown in FIG. 6 may apply more broadly to the measurement of other molecules of interest.

At step 602, apply a relatively high potential to the interference zapping layer 302. At the high potential, the interference zapping layer 302 can oxidize both the electrochemical interferents and the molecule of interest, for example glucose. In aspects where the molecule of interest is glucose, the high potential may be approximately +1.0 V vs. Ag/AgCl. Oxidation as a result of step 602 may deplete the electrochemical interferents and molecules of interest in a region near electrodes 506a and 506b. At step 602, a potential may also be applied to electrodes Pt_{B} and Pt_{F}. In aspects where glucose is the molecule of interest, the potential may applied to electrodes Pt_{B} and Pt_{F} may be +0.6 V.

At step 604, measure current at the working electrodes Pt_{B} and Pt_{F}. This step involves measuring the current at electrodes 506a (Pt_{E}) and 506b (Pt_{B}). Because interferents and molecules of interest may have been oxidized (and thereby eliminated) in the step 602, the background currents of each of the electrodes 506a and 506b—denoted as B(Pt_{E}) and B(Pt_{B}), respectively—may be measurable at this time. It may be desirable to carry out the step of block 604 soon after the step of block 602, such that there is not sufficient time for electrochemical interferents and molecules of interest to diffuse from body fluid 504 exterior to the sensor to the electrodes 506a and 506b.

At step 606, set the interference zapping layer 302 to a low potential. For examples where glucose is the molecule of interest, the interference zapping layer 302 may be set to +0.6 V vs. Ag/AgCl. At such a potential, electrochemical interferents may be oxidized but the molecule of interest, for example glucose, may not be oxidized. At such a potential, the molecule of interest, for example glucose, can diffuse to electrodes 506a and 506b.

At step 608, measure current at electrodes Pt_{B} and Pt_{E}. When the interference zapping layer 302 is set to a relatively low potential, current at the electrode 506b without enzyme layer, I(Pt_{B}), can be measured over time to estimate current due to any interferents that are not blocked by interference zapping layer 302. If the interference zapping layer 302 blocks a relatively high proportion of and/or all of the electrochemical interferents, I(Pt_{B}) may be similar to the value of B(Pt_{B}). I(Pt_{E}), the current at the electrode 506a with enzyme layer 206, is also monitored over time. I(Pt_{B}) signal may mostly be due to concentration of the molecule of interest, for example glucose. A background shift can be estimated by subtracting B(Pt_{B}) from I(Pt_{B}).

At step 610, calculate the current due to the molecule of interest. Current measurements B(Pt_{B}), B(Pt_{E}), I(Pt_{B}), and I(Pt_{E}) generated from the measurement steps 604 and 608 may be used to estimate signal due to the molecule of interest, for example glucose. The current signal due to the molecule of interest Iᵢₙₜ may be equal to the current at electrode 506a (I(Pt_{E}) 610), less the background current of electrode 506a (B(Pt_{E}) 616), less the current of the electrode 506b (I(Pt_{B}) 612), plus the background current of electrode 506b (B(Pt_{B})). Equation 1 shows this mathematical relationship:

I(Pt_{E})−B(Pt_{E})−I(Pt_{B})+B(Pt_{B})=Iᵢₙₜ Equation 1

In some implementations, though measurements of currents I(Pt_{E}) and I(Pt_{B}) at electrodes 506a and 506b, respectively, may be continuous and/or relatively frequent, the measurements of B(Pt_{E}) and B(Pt_{B}) may be less frequent. For example, measurements of B(Pt_{E}) and B(Pt_{B}) may be taken at least once a week, at least once a day, at least twice a day, at least once an hour, at least twice an hour, at least once every ten minutes, at least once every minute or any value or range within or bounded by any of these ranges or values, although values outside these values or ranges can be used in some cases.

The method illustrated in FIG. 6 may also be executed with sensors other than the sensor diagrammed in FIG. 5A. For example, for a sensor such as the sensor illustrated in FIG. 5B, I(Pt_{E}) and B(Pt_{E}) measurements may be made by averaging currents at the two electrodes 506a. The electrode 506b may be used to generate I(Pt_{B}) and B(Pt_{B}). As in the method diagrammed in FIG. 6, an estimate of the signal due to molecule of interest Iᵢₙₜ may be made in accordance with Equation 1. Advantageously, the sensor of FIG. 5B may eliminate, minimize, and/or reduce any error due to concentration gradient of glucose and other species along the length of the sensor.

The method illustrated in FIG. 6 may be executed with the sensor illustrated in FIG. 5C. For example, the two zapping electrodes 514a and 514b may each be used in accordance with step 602 to remove the molecule of interest, for example glucose, and electrochemical interferents. In some implementations, the first interference zapping layer 514a can be used to oxidize the molecule of interest, for example glucose, and the second interference zapping layer 514b can be used to oxidize electrochemical interferents. In other implementations, first interference zapping layer 514a can be used to oxidize electrochemical interferents and second interference zapping layer 514b can be used to oxidize the molecule of interest, for example glucose. Advantageously, the sensor of FIG. 5C can oxidize glucose molecules via interference zapping layers 514a and 514b at a lower potential than interference zapping layer 302 in the example sensor illustrated in FIG. 5A. This can reduce any electrochemical and/or oxidative damage, especially during long sensor sessions, to the polymeric materials in contact with the zapping layers.

Features of a Sensor with an Interference Zapping Layer

### Higher Signal-to-Noise Ratio

Blocking layers, (e.g. layers that block electrochemical interferents based on size or charge) may unintentionally block a target molecule, for example hydrogen peroxide. About 20-30% of desired signal from hydrogen peroxide created from enzymatic conversion of glucose may be blocked by a size-based blocking layer underlying an enzyme layer. It is possible that charge-based blocking layers may also result in a similar reduction in signal due to blocking of the target molecule.

Advantageously, a sensor including an interference zapping layer according to the present disclosure may avoid a similar reduction in signal due to blocking of the target molecule. For a sensor including an interference zapping layer, the hydrogen peroxide generated at the enzyme layer as a byproduct of glucose conversion may have unimpeded, substantially unimpeded, and/or minimally impeded access to the working electrode. Implementation of such an interference zapping layer may result in at least a 20 to 30% increase in glucose-proportional current on the working electrode. For target molecules other than hydrogen peroxide, the increase in signal afforded by implementation of an interference zapping layer may vary depending on the target molecule's diffusion characteristics and the distance from the enzyme layer to the working electrode. Such an increase in signal, while noise remains at the same and/or a similar level, may result in an improved signal-to-noise ratio.

### Reduced Size of Sensor

An increased S/N ratio may allow for miniaturizing the sensor from 300 μm in length to a smaller size. Decreasing the size of the sensor may be desirable. For example, a small sensor may be less likely to cause pain to a patient. Reducing pain during usage may improve the patient experience and/or broaden the population of people on which a sensor may be used. As an illustrative example, children may have low pain tolerances relative to adults, so miniaturizing the sensor may allow more children to tolerate the sensor.

### Increased Sensor Longevity

Certain molecules of interest, for example hydrogen peroxide, may be highly reactive. Hydrogen peroxide belongs to a class of molecules called reactive oxygen species (ROS). ROS may react with many different biological molecules, including proteins. Enzymes, for example glucose oxidase enzyme, are proteins. Prolonged exposure to a ROS may degrade an enzyme's activity. For example, prolonged exposure of hydrogen peroxide may degrade glucose oxidase's ability to convert glucose. It may be desirable to prevent and/or minimize an enzyme's exposure to ROS in order to extend a sensor's operating life.

In an implementation of a glucose sensor in accordance with the sensor of FIG. 2, the hydrogen peroxide generated via the enzymatic conversion of glucose diffuses outward and inward from the enzyme layer 208. The inward diffusing hydrogen peroxide molecules may encounter the working electrode 202, where they can be converted to benign molecules, such as oxygen (O₂) and water (H₂O). However, the hydrogen peroxide molecules that do not encounter the working electrode 202 may linger within the sensor, where they may oxidatively degrade sensor components, including the enzyme within the enzyme layer. This oxidative reaction may contribute to the relatively short operating lifespan of a glucose sensor of this type, which, in some cases, may be approximately 10 days.

Advantageously, the implementation according to this disclosure may address the problem of ROS degradation of components of the sensor. In some examples, such as those illustrated in FIGS. 3A-3C, the interference zapping layer 302, which encircles the enzyme layer 208, can contact ROS, such as hydrogen peroxide, and subsequently oxidize them, for example converting hydrogen peroxide to oxygen and water. In this way, the amount of ROS, for example hydrogen peroxide, within the sensor can be lowered, thereby reducing the effect of ROS-mediated degradation of sensor components.

### Sensor Calibration In Vivo

In vitro calibration of an electrochemical sensor, for example a glucose electrochemical sensor, may involve measuring the sensor signals in 0 mg/dL of the molecule of interest, for example glucose, before exposing the sensor to various other concentrations to obtain the calibration plot. For an example CGM, this “true” baseline measurement at 0 mg/dL glucose can be subtracted from each subsequent signal to estimate the true concentration of glucose in the test fluid more accurately.

Such a controlled experiment as the “true” baseline measurement may not be possible in vivo. There may never, essentially never, or rarely be a 0 mg/dL glucose concentration in the biofluids surrounding the sensor. Hence, in vivo the sensor may be exposed to a certain level of glucose concentration, making it difficult to estimate the true baseline current value of the sensor.

Generally, a baseline estimate can be made from conducting in vitro measurements using a sample of sensors from a manufactured batch of sensors. This baseline estimate can be subsequently applied to correct in vivo glucose estimates of other sensors in that batch. However, true background current can be different between electrodes of the same batch. This difference may be attributable to several factors, including the roughness of the electrode and/or variation in composition of the biofluid compared to the testing solution used during in vitro experiments. Thus, using a mean background value from a set of sample electrodes from a particular batch can still lead to erroneous glucose estimates.

Certain aspects of the present disclosure may overcome these issues. For example, the interference zapping layer may be used to eliminate glucose in the vicinity of the working electrode, which can allow for measuring a baseline while the sensor is situated in vivo.

### Correction for Sensor Drift

The number and type of interfering molecules may vary by subject. Other factors that may influence the number and the type of interfering molecules to which a sensor may be exposed include the depth at which the sensor is implanted, hydration level, exercise, vitamin C intake, acetaminophen intake, local inflammation, etc. However, these changes may occur on a relatively short timescale of several hours, before the site of probe insertion equilibrates to a steady state.

It may be possible to account for the degradation of the sensor's outer layer and occlusion of the sensor as a result of inflammation response to implanting the sensor. In certain aspects of the present disclosure, determining the effect of degradation and/or occlusion of the sensor's outer layer may be accomplished by measuring the steady-state interference level and by quantifying the current passing through the interference zapping layer over the timescale of several hours to a day. Contrastingly, the degradation and occlusion of the sensor may occur in the timescale of days. The daily relative decrease or increase in the interference levels compared to the first day of sensor implantation can be used to correct the amount of glucose available to the sensor. This measurement can be used by a hardware processor to correct for sensor drift and potentially increase the accuracy of glucose measurements over the entire working lifetime of the sensor.

Other aspects of a sensor in accordance with the present disclosure allow for making a baseline measurement at any time during the sensor session, which may last for several days. In these conditions, the sensor baseline may shift in unpredictable ways, possibly in response to degradation of sensor elements during operation and/or to change in the properties of electrode surface, for example metal of the electrode slowly changing to various oxides of that metal. By measuring a baseline value at various time points during the sensor session, the drift in baseline can be monitored and the glucose estimation can be corrected accordingly.

In addition, the methods discussed herein may eliminate the necessity to develop factory calibrations based on extensive data collection and provide an efficient means to perform device corrections. Advantageously, elimination of factory calibrations may save time in sensor manufacturing.

### Increase Oxygen Availability at Enzyme Layer

As described previously, the interference zapping layer may oxidize hydrogen peroxide to generate oxygen and water. The generated oxygen may diffuse to the enzyme layer, increasing oxygen availability for glucose conversion. Thus, a sensor constructed in accordance with the present disclosure may advantageously increase oxygen at the enzyme layer to excess, such that the glucose conversion reaction as illustrated by FIG. 1 may be glucose-limited, rather than being oxygen-limited.

### Modulation by Voltage

Because, in some aspects, the interference zapping layer may be physically separated from the electrode, the present disclosure allows for independent control of unwanted interference oxidation using voltage. Such control may allow the development of unique measurement strategies and/or protocols for accurate glucose sensing, as discussed in the following examples.

### Reduction of Hysteresis

Hysteresis is a phenomenon by which a time-evolving state retains “memory” of an earlier state, which adds bias to a measurement of the present state. In the context of electrode-based glucose measurement, hysteresis may be exaggerated when the glucose level of a person drops steeply (due to, for example, an insulin bolus and/or exercise, etc.). The hydrogen peroxide generated a few minutes before the current measurement, while glucose was still at a high level of concentration, may linger within the sensor and artificially increase the glucose measurement.

A sensor in accordance with the present disclosure may be able to reduce the effect of hysteresis. For example, applying a potential to the interference zapping layer for a given amount of time while the working electrode is inactive (e.g., no potential is applied to the working electrode) may oxidize all, substantially all, or a many of the residual target molecules, for example hydrogen peroxide, from the previous state. After a certain length of zapping layer activation time, the working electrode may be activated to determine an unbiased reading, for example a reading of current due to glucose.

### Reduction of Temperature Drift

Generally, enzyme activity may be sensitive to temperature. Glucose oxidase's activity, for example, may increase many folds when transitioning from room temperature to human body temperature, for example upon insertion of the probe into a subject's tissue. An increase in glucose oxidase's activity may translate to a higher current at the glucose sensing electrode simply due to increased throughput of the enzyme.

An example sensor in accordance with the present disclosure may be able to correct for the effect of temperature on the enzyme of the enzyme layer. For example, if the temperature of the sensor and the interference zapping layer current are measured, the throughput of the enzyme of the enzyme layer can be algorithmically interpolated, for example by a hardware processor. The algorithmic interpolation may include comparing empirical measurements of the system at various temperatures and interference zapping layer currents, for example.

Correction for User Food, Beverage, and/or Drug Intake-Based Interference

Various electrochemical interferents may have different oxidation potentials, as previously discussed. If the user takes a certain food, beverage, or drug, for example orange juice or acetaminophen, the interference zapping layer may be set to a specific potential to oxidize an electrochemical interferent introduced to the user by that food, beverage, or drug. The interference zapping layer potential may be tuned to account for a user's physiology and/or diet. The determination to tune the interference zapping layer potential in response to food, beverage, and/or drug intake can be made by, for example, a lifestyle management app in combination with the glucose sensor. As an illustrative example, the sensor may be used in conjunction with Nudge, a Cercacor lifestyle management app.

### Sensor Capable of Rapid Hydration

Optionally, inclusion of a hydrophilic polymer in a sensor in accordance with the present disclosure may be desirable to ensure rapid wetting and/or hydration of the sensor. For example, a hydrophilic layer of the sensor may include a hydrophilic polymer. Rapid wetting and/or hydration of the sensor may result in faster sensor stabilization, reducing a “warm-up” time after the sensor has been inserted. Rapid wetting and/or hydration may also result in lower impedance. Rapid wetting and/or hydration may help an enzyme of the enzyme layer increase activity. Rapid wetting and/or hydration may help an enzyme of the enzyme layer reach maximum and/or optimal activity.

Peel-off and/or cracking of certain hydrophilic polymer layers may occur during long sensor sessions. For example, peel-off and/or cracking of certain hydrophilic polymer layers may occur during sensor sessions lasting hours and/or days. Without being bound to a particular theory, it is believed that peel-off and/or cracking of a hydrophilic polymer layer may be due to swelling of the hydrophilic polymers and decreasing adhesion between the hydrophilic polymer layer and surrounding sensor layers, for example the interference zapping layer, the blocking layer, the enzyme layer, and/or the analyte limiting layer. Peel-off and/or cracking may result in non-uniformity in the interference blocking layer, which may thereby allow electrochemical interferents to reach the electrode. To ameliorate these issues and shorten wetting time, a sensor may include a wetting layer including a hydrophilic polymer.

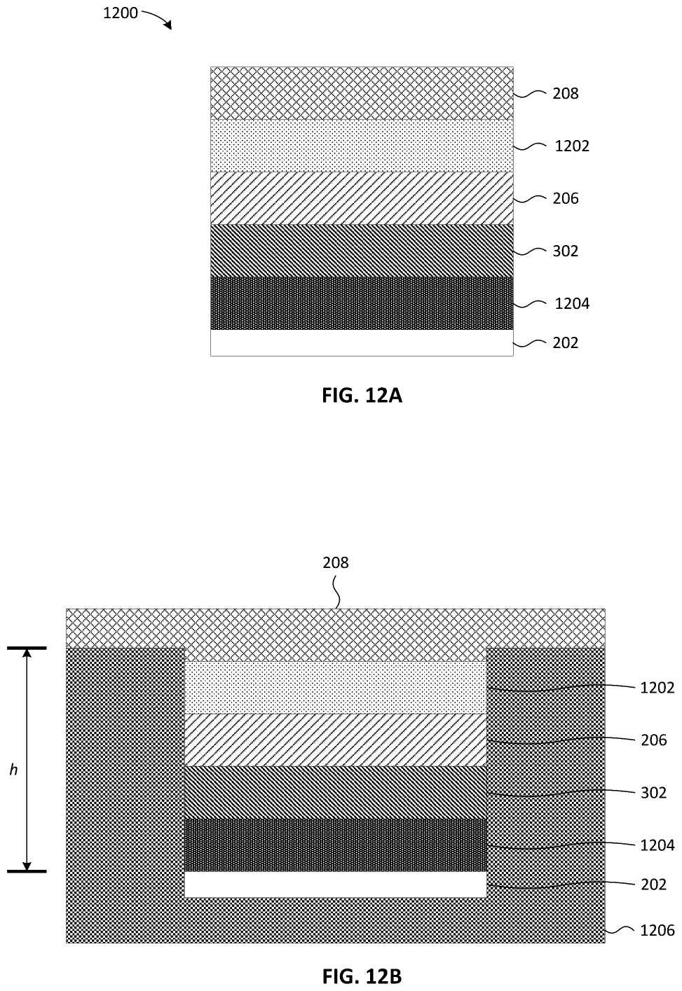

Now with reference to FIG. 12A, a sensor 1200 adapted for rapid hydration may include an analyte limiting layer 208, an adhesion promoter layer 1202, an enzyme layer 206, an interference zapping layer 302, a wetting layer 1204, and an electrode 202. The analyte limiting layer 208, the enzyme layer 206, the interference zapping layer 302, and the electrode 202 may each be implemented in accordance with the present disclosure. In some aspects, the analyte limiting layer 208 may include a polyurethane. In some aspects, the adhesion promoting layer 1202 may include a hydrophilic material, for example a hydrophilic polyurethane. The electrode 202 may include platinum.

In some aspects, the wetting layer 1204 may include a doped polyurethane. In some aspects, the polyurethane is doped with a co-polymer polyvinylpyrrolidone-co-polyvinyl acetate (PVP-co-PVAc) copolymer. In some aspects, the wetting layer 1204 includes polyvinypyrrolidone (PVP). In some aspects, the wetting layer 1204 may include a sulfonated tetrafluoroethylene based fluorpolymer-copolymer (e.g. Nafion™). Inclusion of a wetting layer 1204 may inhibit, prevent, and/or minimize peel-off and/or layer cracking. Hydrophilic polyurethane materials may be capable of absorbing up to 30% water by mass when exposed to aqueous solution. The rate of water absorption of a non-doped hydrophilic polyurethane material may be slow, which, if included in a sensor, can lead to high impedance for the sensor and lower transmission of target molecules (e.g. hydrogen peroxide) to the electrode 202 shortly after insertion of the sensor to a patient's body (e.g. within minutes to hours of insertion). Inclusion of a dopant, for example PVP-co-PVAc copolymer, in the wetting layer 1204 may increase rate at which the wetting layer 1204 can absorb water. Advantageously, both the hydrophilic polyurethane and the PVP-co-PVAc co-polymer are soluble in organic solvents, and thus fabrication of a wetting layer 1204 in accordance with the present aspect can include few steps. Other suitable materials providing sufficient adhesion and water absorbance may also be suitable for inclusion in the wetting layer 1204.

In some aspects, the enzyme layer 206 may include a waterborne polyurethane, a polyethylene glycol diglycidyl ether, and/or a polyethylene diamine. In some aspects, the enzyme layer 206 may include glucose oxidase. The enzyme layer 206 may be formed using a water-borne polyurethane material and a cross-linked polyethylene diamine. Without being bound to a particular theory, it is believed that the presence of amines (e.g. polyethylene diamine) within the enzyme layer 206 provide a positive charge, allowing the enzyme layer 206 to rapidly hydrate. In aspects where the enzyme layer 206 includes a polyurethane matrix, the polyurethane matrix may provide adhesion between the enzyme layer 206 and other polyurethane-based layers, such as the analyte limiting layer 208. Other suitable materials providing sufficient adhesion and water absorbance may also be suitable for inclusion in the enzyme layer 206.

Now with reference to FIG. 12B, a sensor including a wetting layer may include an analyte limiting layer 208, an adhesion promoter layer 1202, an enzyme layer 206, an interference zapping layer 302, a wetting layer 1204, an electrode 202, and an insulator 1206. The analyte limiting layer 208, the adhesion promoter layer 1202, the enzyme layer 206, the interference zapping layer 302, the wetting layer 1204, and the electrode 202 may all be implemented in accordance with the present disclosure. As depicted in FIG. 12B, the insulator 1206 may encompass the adhesion promoter layer 1202, the enzyme layer 206, the interference zapping layer 302, the wetting layer 1204, and the electrode 202. In such an aspect, when the sensor is in contact with a patient's body fluid (e.g., interstitial fluid), the insulating material may ensure that the body fluid can only contact the electrode 202 via the layers 208, 1202, 206, 302, and 1204.

In some aspects, the height h between the surface of the analyte limiting layer 208 and the surface of the electrode 202 may be about 5, 6, 7, 8, 9, 10, 11, 12, 13, 14, 15, 16, 17, 18, 19, 20, 21, 22, 23, 24, 25, 26, 27, 28, 29, or 30 μm, or within a range defined by any of the preceding values. In some aspects, the height h is from about 10 to about 15 μm.

In some aspects, the analyte limiting layer 208 may be about 1, 2, 3, 4, 5, 6, 7, 8, 9, 10, 11, 12, 13, 14, or 15 μm in thickness, or within a range defined by any of the preceding values. In some aspects, the thickness of analyte limiting layer 208 may be from about 2 to about 10 μm.

In some aspects, the adhesion promoting layer 1202 may be about 0.1, 0.2, 0.4, 0.5, 0.6, 0.7, 0.8, 0.9, 1.0, 1.1, 1.2, 1.3, 1.4, or 1.5 μm in thickness, or within a range defined by any of the preceding values. In some aspects, the thickness of the adhesion promoting layer 1202 may be from about 0.1 to about 1 μm.

In some aspects, the enzyme layer 206 may be about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 μm in thickness, or within a range defined by any of the preceding values. In some aspects, the thickness of the enzyme layer 206 may be from about 1 to about 5 μm.

In some aspects, the interference blocking layer 302 may be about 0.5, 1.0, 1.5, 2.0, 2.5, 3.0, 3.5, 4.0, 4.5, 5.0, 5.5, 6.0, 6.5, 7.0, or 7.5 μm in thickness, or within a range defined by any of the preceding values. In some aspects, the thickness of the interference blocking layer 302 may be from about 2 to about 5 μm.

In some aspects, the wetting layer 1204 may be about 0.1, 0.2, 0.3, 0.4, 0.5, 0.7, 0.8, 0.9, 1.0, 1.1, 1.2, 1.3, 1.4, or 1.5 μm in thickness, or within a range defined by any of the preceding values. In some aspects, the thickness of the wetting layer 1204 may be from about 0.1 to about 1 μm.

### Measurement Mode

In some aspects, a sensor in accordance with the present disclosure can execute a measurement mode where, during a measurement, the sensor may proceed through a plurality combinations of working electrode potential and zapping layer potential. Thus, the working electrode potential can be sampled at a plurality of potentials at each of a plurality of zapping layer potentials. Such a sensor may thereby generate a rich data set, which may allow a hardware processor to determine the concentration of one or more physiological analytes, including, for example, glucose. For example, the sensor may make a measurement for each combination of working electrode potential and zapping layer potential. Such an approach may render a factory calibration step unnecessary, as background currents, signal due to electrochemical interferents, and signal due to analytes of interest may all be captured as the sensor proceeds through combinations of zapping layer potentials and working electrode potentials. Such an approach may allow for measurement of more molecular species, such as concentration estimates for one or more analytes of interest and/or concentration estimates for electrochemical interferents. Such an approach may allow a device with a sensor of the present disclosure to warn a user of measurement inaccuracy due to electrochemical interferents. A sensor executing a measurement mode may be capable of estimating the concentration of particular electrochemical interferents. Such an approach may allow for measurements that focus on particular combinations of working electrode and interference zapping layer potentials. For example, on a first measurement, it may be possible to identify signals at particular a particular working electrode potential and a particular interfere electrode potential. Subsequent measurements may measure working electrode current at the particular working electrode potential and particular interference zapping layer potential without measuring current at every combination of working electrode potential and interference zapping layer potential.

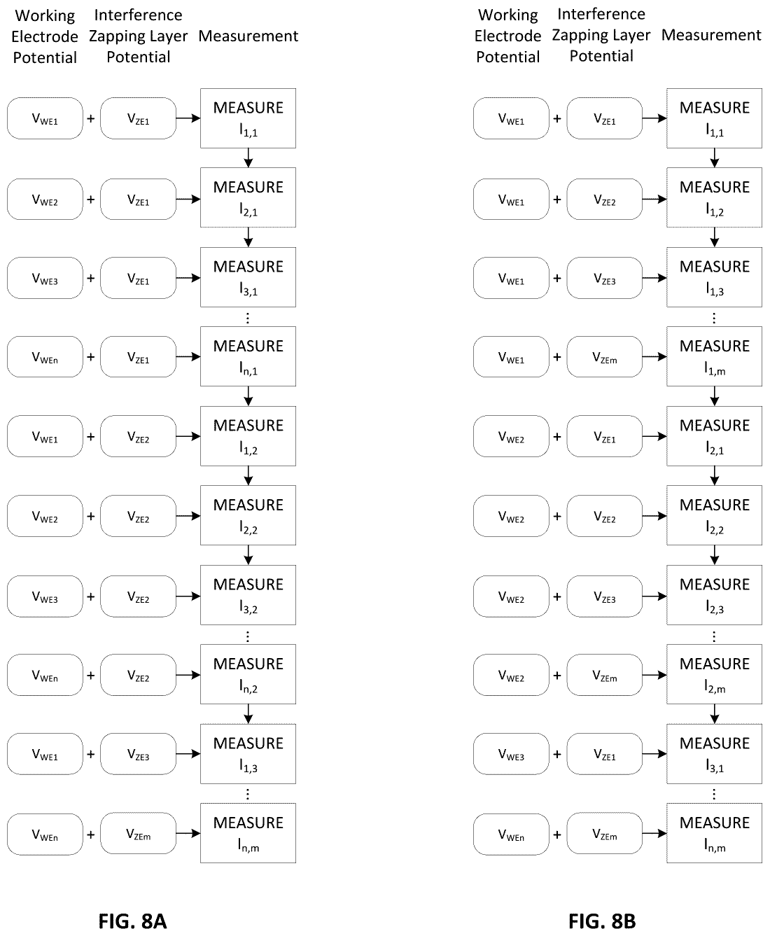

With reference to FIG. 7, a sensor in accordance with the present disclosure may set a potential V_{WE} of its working electrode to a plurality of different voltages, including, for example V_{WE1}, V_{WE2}, V_{WE3}, V_{WE4}, through V_{WEn} Consecutive working electrode potential values (e.g., V_{WE1} and V_{WE2}) may be separated by a difference of ΔV_{WE}. In some aspects, the difference ΔV_{WE} may be equal across the range of voltages from V_{WE1} to V_{WEn}. In other aspects, the difference ΔV_{WE} may be nonuniform and/or varied across the range of voltages from V_{WE1} to V_{WEm}. The sensor in accordance with the present disclosure may also set a potential V_{ZE} of its interference zapping layer to a plurality of different voltages, including, for example, V_{ZE1}, V_{ZE2}, V_{ZE3}, V_{ZE4}, through V_{ZEm} Consecutive zapping layer potential values (e.g., V_{ZE1} and V_{ZE2}) may be separated by a difference of ΔV_{ZE}. In some aspects, the difference ΔV_{ZE} may be equal across the range of voltages from V_{ZE1} to V_{ZEm}. In other aspects, the difference ΔV_{ZE} may be nonuniform and/or varied across the range of voltages from V_{ZE1} to V_{ZEm}. The sensor may generate a discrete measurement I_{x,y} of current while the working electrode potential is set to a voltage V_{WEx} and while the interference zapping layer is set to a voltage V_{ZEy}. For instance, when the working electrode potential is set to V_{WE1} and the zapping layer potential is set to V_{ZE1}, discrete measurement I_{1,1} can be generated. As a further example, when the working electrode potential is set to V_{WE2} and the zapping layer potential is set to V_{ZE1}, discrete measurement I_{2,1} can be generated. As a further example, when the working electrode potential is set to V_{WE1} and the zapping layer potential is set to V_{ZE2}, discrete measurement I_{1,2} can be generated. As a further example, when the working electrode potential is set to V_{WEn} and the zapping layer potential is set to V_{ZEm}, discrete measurement I_{n,m} can be generated. The measurements can be stored and/or represented as a matrix, for example matrix I_{1-n,1-m}, as depicted in FIG. 7.

As the voltage V_{ZE} of the interference zapping layer changes, certain physiological analytes may be oxidized before they can encounter the working electrode, for example by diffusion. Accordingly, as the potential V_{ZE} of the zapping layer changes, the current measured by the working electrode due to detectable analytes may change accordingly.

As the voltage of the working electrode V_{WE} varies, the measured current may depend on the voltametric profiles of analytes being oxidized by the working electrode at potential V_{WE}.

The working electrode potential V_{WE} may be set to a variety of different voltages as part of a measurement mode in accordance with the present disclosure. In some aspects, the potential V_{WE} may be set to one or more voltages ranging from of about 0 V Ag/AgCl, +0.01 V Ag/AgCl, +0.05 V Ag/AgCl, +0.1 V Ag/AgCl, +0.2 V Ag/AgCl, +0.3 V Ag/AgCl, +0.4 V Ag/AgCl, +0.5 V Ag/AgCl, +0.6 V Ag/AgCl, +0.7 V Ag/AgCl, +0.8 V Ag/AgCl, +0.9 V Ag/AgCl, +1.0 V Ag/AgCl, +1.1 V Ag/AgCl, +1.2 V Ag/AgCl, +1.3 V Ag/AgCl, +1.4 V Ag/AgCl, +1.5 V Ag/AgCl, +1.6 V Ag/AgCl, +1.7 V Ag/AgCl, +1.8 V Ag/AgCl, +1.9 V Ag/AgCl, or +2.0 V Ag/AgCl, or in a range defined by any two of the preceding values. In some aspects, the range of working electrode potentials may be about 0 V to +1.5 V Ag/AgCl, 0 V to +1.25 V Ag/AgCl, 0 V to +1.1 V Ag/AgCl, 0 V to +1.0 V Ag/AgCl, or 0 V to +0.7 V Ag/AgCl. In some aspects, the working electrode potential V_{WE} may be set to a series of voltages having a ΔV_{WE} of 0.001 V, ΔV_{WE} of 0.01 V, a ΔV_{WE} of 0.05 V, a ΔV_{WE} of 0.1 V, a ΔV_{WE} of 0.1 V, a ΔV_{WE} of 0.2 V, a ΔV_{WE} of 0.25 V, a ΔV_{WE} of 0.3 V, a ΔV_{WE} of 0.4 V, a ΔV_{WE} of 0.5 V, a ΔV_{WE} of 0.75 V, or a ΔV_{WE} of 1 V.

The zapping layer potential V E may be set to a variety of different voltages as part of a measurement mode in accordance with the present disclosure. In some aspects, the potential V_{ZE} may be set to one or more voltages ranging from of about 0 V Ag/AgCl, +0.01 V Ag/AgCl, +0.05 V Ag/AgCl, +0.1 V Ag/AgCl, +0.2 V Ag/AgCl, +0.3 V Ag/AgCl, +0.4 V Ag/AgCl, +0.5 V Ag/AgCl, +0.6 V Ag/AgCl, +0.7 V Ag/AgCl, +0.8 V Ag/AgCl, +0.9 V Ag/AgCl, +1.0 V Ag/AgCl, +1.1 V Ag/AgCl, +1.2 V Ag/AgCl, +1.3 V Ag/AgCl, +1.4 V Ag/AgCl, +1.5 V Ag/AgCl, +1.6 V Ag/AgCl, +1.7 V Ag/AgCl, +1.8 V Ag/AgCl, +1.9 V Ag/AgCl, or +2.0 V Ag/AgCl, or in a range defined by any two of the preceding values. In some aspects, the range of interference zapping layer potentials may be about 0 V to +1.5 V Ag/AgCl, 0 V to +1.25 V Ag/AgCl, 0 V to +1.1 V Ag/AgCl, 0 V to +1.0 V Ag/AgCl, or 0 V to +0.7 V Ag/AgCl. In some aspects, the interference zapping layer potentials V E may be set to a series of voltages having a ΔV_{ZE} of 0.001 V, ΔV_{ZE} of 0.01 V, a ΔV_{ZE} of 0.05 V, a ΔV_{ZE} of 0.1 V, a ΔV_{ZE} of 0.1 V, a ΔV_{ZE} of 0.2 V, a ΔV_{ZE} of 0.25 V, a ΔV_{ZE} of 0.3 V, a ΔV_{ZE} of 0.4 V, a ΔV_{ZE} of 0.5 V, a ΔV_{ZE} of 0.75 V, or a 0.4 ΔV_{ZE} of 1 V.

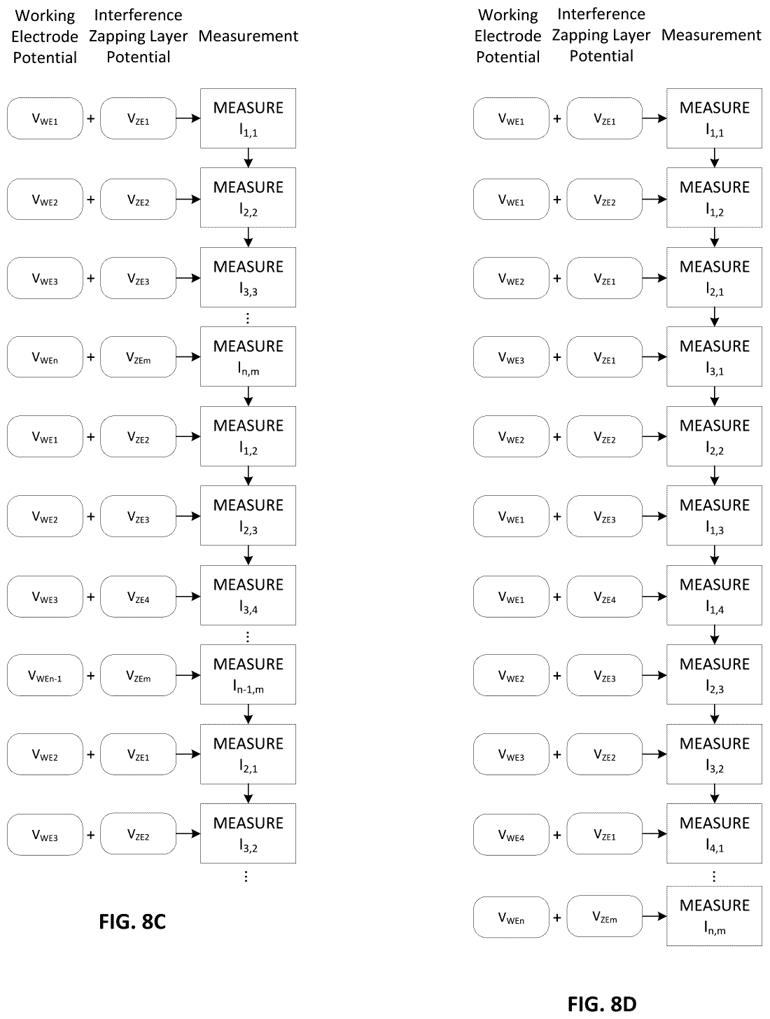

The working electrode potential and zapping layer potentials may be set simultaneously in a sequence such that all or a subset of currents of matrix I_{1-n,1-m} are measured. The working electrode potential and zapping layer potentials may be set in any suitable sequence. In some aspects, while the interference zapping layer may be held at a particular potential, the potential applied to the working electrode may proceed through a series of potentials. For example, with reference to FIG. 8A, the interference zapping layer may be held at potential V_{ZE1} as the working electrode potential proceeds, for example by stepping, from V_{WE1} to V_{WE2}, from V_{WE2} to V_{WE3}, etc. through V_{WEn} before the interference zapping layer is set to potential V_{ZE2} and working electrode potential proceeds again from V_{WE1} through V_{WEn}, etc as shown. It is to be understood that, though the working electrode potential is shown as increasing in FIG. 8A, the working electrode potential may alternatively proceed by decreasing, for example from V_{WE4} to V_{WE3}, from V_{WE3} to V_{WE2}, etc. It is to be understood that, though the zapping interference is shown as increasing in FIG. 8A, the zapping interference layer may alternatively proceed by decreasing, for example from V_{ZE4} to V_{ZE3}, from V_{ZE3} to V_{ZE2}, etc.

In some aspects, while the working electrode may be held at a particular potential, the potential applied to the interference zapping layer may proceed through the first plurality of potentials. For example, with reference to FIG. 8B, the working electrode may be held at potential V_{WE1} as the interference zapping layer proceeds, for example by stepping, from V_{ZE1} to V_{ZE2}, from V_{ZE2} to V_{ZE3}, through V_{ZE3} before the working electrode is set to V_{WE2} before the zapping layer potential proceeds again from V_{ZE1} through V_{ZE3} etc. as shown. It is to be understood that, though the working electrode potential is shown as increasing in FIG. 8B, the working electrode potential may alternatively proceed by decreasing, for example from V_{WE4} to V_{WE3}, from V_{WE3} to V_{WE2}, etc. It is to be understood that, though the zapping interference is shown as increasing in FIG. 8B, the zapping interference layer may alternatively proceed by decreasing, for example from V_{ZE4} to V_{ZE3}, from V_{ZE3} to V_{ZE2}, etc. In some aspects, each of the potential of the working electrode and the potential of the zapping layer may change for successive measurements made by the working electrode. For example, with reference to FIG. 8C, the working electrode potential may be set to V_{WE1} while the interference zapping layer may be set to V_{ZE1} to measure I_{1,1}. Then, the working electrode potential may be set to V_{WE2} and the interference zapping layer may be set to V_{ZE2} to measure I_{2,2}. Then, the working electrode potential may be set to V_{WE3} and the interference zapping layer may be set to V_{ZE3} to measure I_{3,3}, etc. For example, with reference to FIG. 8D, the working electrode potential may be set to V_{WE1} while the interference zapping layer may be set to V_{ZE1}. Then, the working electrode potential may be set to V_{WE1} and the interference zapping layer may be set to V_{ZE2} to measure I_{1,2}. Then, the working electrode potential may be set to V_{WE2} and the interference zapping layer may be set to V_{ZE1} to measure I_{2,1}, etc.

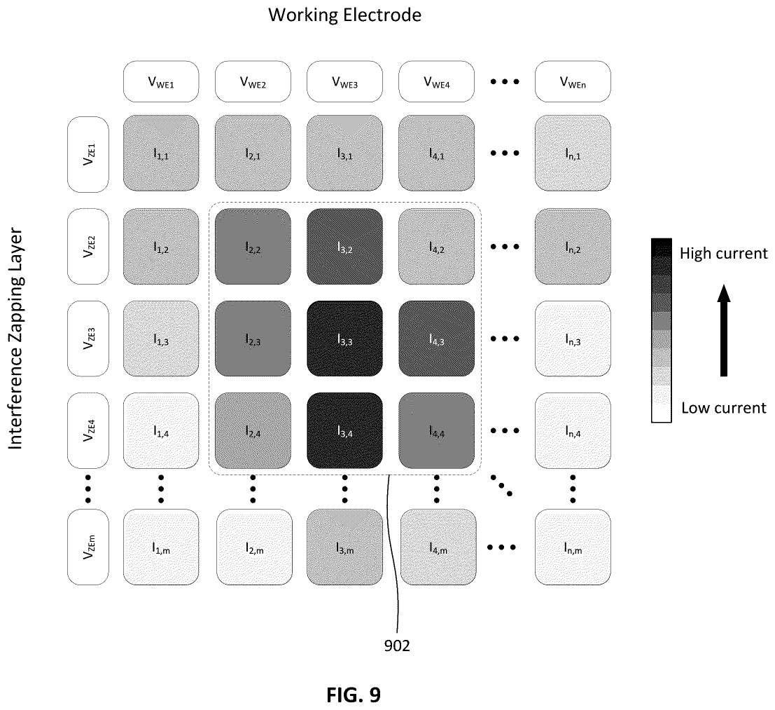

In some aspects, a hardware processor in communication with a sensor as disclosed herein can create a data structure made up of measurements. Creating a data structure may advantageously incorporate some and/or all of the currents measured such that an analyte concentration may be determined with a rich data set, rather than a single working electrode measurement at a single interference zapping layer potential. In some aspects, the measurements constituting matrix I_{1-n,1-m} and/or a subsection of matrix I_{1-n,1-m} may define a heatmap, as shown in FIG. 9. The currents I_{1-n,1-m} may be organized by their respective working electrode potential V E and interference zapping layer potential V_{ZE}, and magnitude of current may be represented by color. A hardware processor may be capable of outputting a heatmap to a display. A hardware processor may be capable of comparing a heatmap measured from a patient to one or more previous heatmaps. For example, a hardware processor may compare a heatmap measured from a patient to a heatmap measured from a subject for whom ISF concentrations of one or more molecular species is known. For example, a hardware processor may compare a heatmap measured from a patient to a heatmap measured from a control fluid where concentration of one or more molecular species is known. For example, a hardware processor may compare a heatmap measured from a patient to a previous heatmap measured from the patient. The processor may thereby be able to estimate concentrations of one or more molecular species. Current matrix I_{1-n,1-m} may be displayed as a three-dimensional plot. For example, height in the z-dimension may correspond to measured current, while position along the x-axis may correspond to working electrode potential V_{WE} and position along the y-axis may correspond to interference zapping layer potential V_{ZE}. Any combination of axes and variables and/or outputs may be used, however. For example, a hardware processor may compare a three-dimensional plot measured from a patient to a three-dimensional plot measured from a subject for whom ISF concentrations of one or more molecular species is known. For example, a hardware processor may compare a three-dimensional plot measured from a patient to a three-dimensional plot measured from a control fluid where concentration of one or more molecular species is known. For example, a hardware processor may compare a three-dimensional plot measured from a patient to a previous three-dimensional plot measured from the patient. The processor may thereby be able to estimate concentrations of one or more molecular species. Current matrix I_{1-n,1-m} may be displayed as an array. For example, values in the array may correspond to measured current, while position within the array may correspond to working electrode potential V_{WE} and/or to interference zapping layer potential V_{ZE} For example, a hardware processor may compare an array measured from a patient to array measured from a subject for whom ISF concentrations of one or more molecular species is known. For example, a hardware processor may compare an array measured from a patient to array measured from a control fluid where concentration of one or more molecular species is known. For example, a hardware processor may compare an array measured from a patient to a previous array measured from the patient. The processor may thereby be able to estimate concentrations of one or more molecular species. The processor may use any suitable data structure and/or representation of the measurements to make comparison to control measurements (e.g. measurements from other subjects or measurements from fluids having known quantities of analyte) and/or previous patient measurements.

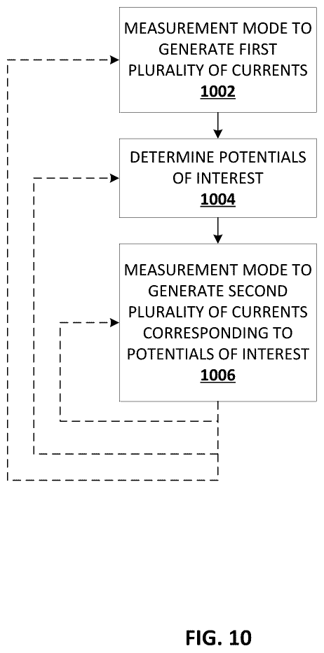

In some aspects, during measurement mode, only a subset of the measurement matrix I_{1-n,1-m} depicted in FIG. 7 may be generated. FIG. 10 diagrams an example process where a subset of measurement matrix I_{1-n,1-m} is measured. In step 1002, the sensor executes a measurement mode to measure a first plurality of currents. The first plurality of currents may be, for example, the entire current matrix I_{1-n,1-m}. In step 1004, a processor may determine a range of potentials of interest, for example a set and/or range of working electrode potentials V_{WE} and/or a set and/or range of zapping interference layer potentials V_{ZE} The potentials of interest may correspond to a second plurality of currents. A processor may determine the potentials of interest based at least in part on the first plurality of currents. For example, now with reference to FIG. 9, the processor may identify a subset 902 of currents within matrix I_{1-n,1-m} that may be indicative of the concentration of an analyte and/or a molecular species. As an illustrative example, subset 902 may have relatively high currents due to oxidation of a molecular species. Though subset 902 is depicted as corresponding to a contiguous set of interference zapping layer potentials (i.e. V_{ZE2}-V_{ZE4}) and a contiguous set of working electrode potentials (i.e. V_{WE2}-V_{WE4}), potentials of interest identified in step 1004 need not be contiguous. In some aspects, the hardware processor may determine potentials of interest, omitting potentials that are irrelevant and/or minimally relevant to molecular species to be monitored. In step 1006, the sensor may enter measurement mode to generate a second plurality of currents. The second plurality of currents may correspond to the potentials of interest determined by the processor. The second plurality of currents may include fewer measured currents than the first plurality of currents. Measuring a subset of the current matrix I_{1-n,1-m} may be desirable, for example, to decrease a time to measurement by an electrochemical probe in accordance with the present disclosure. Because step 1006 involves selectively measuring current at only certain potentials, step 1006 may require less time to complete than step 1002. Likewise, completion of step 1006 may require less energy input than completion of step 1002 because step 1006 involves fewer measurements.

Steps 1002, 1004, and 1006 may be optionally periodically repeated. Step 1006 may be periodically performed, for example, to identify changes in concentration of one or more molecular species of interest. It may be desirable to repeat step 1006 when a measurement is desired but a full current matrix I_{1-n,1-m} is not necessary. Step 1006 may be repeated when, for example, a detailed measurement is unnecessary, minimized sensor power output is desired, and/or minimized time to measurement completion is desired. It may be desirable to repeat step 1002 if power output and/or time to measurement completion is not a concern. It may be desirable to repeat step 1002 to re-measure currents corresponding to potentials beyond those identified as potentials of interest. It may be desirable to repeat step 1004 in response to new measurements made in either of steps 1002 and/or 1006. In some aspects, step 1002 may be repeated less frequently than step 1006. In some aspects, step 1002 may be repeated at the same frequency as step 1006. In some aspects, step 1002 may be repeated more frequently than step 1006. In some aspects, the frequencies of steps 1002 and/or 1006 repeating may at least in part on depend on measured currents. In some aspects, step 1004 is repeated no more frequently than either step 1002 and/or step 1006. In some aspects, step 1002 may be repeated at least once every week, day, hour, half hour, 10 minutes, 1 minute, 30 seconds, 10 seconds, 1 second, or in in a range defined by any two of the proceeding values. In some aspects, step 1004 may be repeated at least once every week, day, hour, half hour, 10 minutes, 1 minute, 30 seconds, 10 seconds, 1 second, or in in a range defined by any two of the proceeding values. In some aspects, step 1006 may be repeated at least once every week, day, hour, half hour, 10 minutes, 1 minute, 30 seconds, 10 seconds, 1 second, or in in a range defined by any two of the proceeding values.

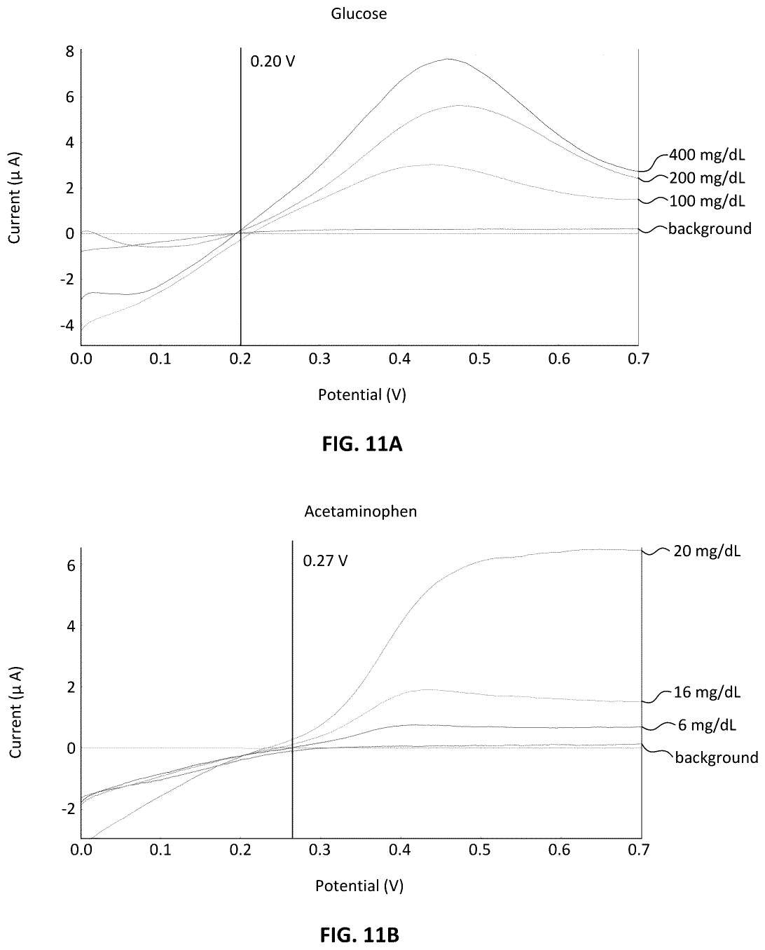

Advantageously, a sensor capable of executing a measurement mode in accordance with the present disclosure may be capable of deconvoluting current signals to identify contributions by individual molecular species or groups of molecular species. FIG. 11A is a cyclic voltammogram, plotting detected current as a function of working electrode potential V_{WE} for various concentrations of glucose in vitro. Current I_{glu} may be a function ƒ(V_{WE}, c_{glu}) of working electrode potential V_{WE} and glucose concentration c_{glu}. FIG. 11B is a cyclic voltammogram, plotting detected current as a function of working electrode potential V_{WE} for various concentrations of acetaminophen. Glucose and acetaminophen are each an example molecular species that may be detectable by a measurement mode, and FIGS. 11A-11B are provided as illustrative examples of voltametric profiles of such molecular species. Current I_{ace} may be a function ƒ(V_{WE}, c_{ace}) of working electrode potential V_{WE} and acetaminophen concentration c ace. For an electrochemical measurement of a patient's ISF, a resulting voltammogram may be a convolution of a plurality of current signals across the potential range measured by a working electrode, as discussed herein. Measured current Iₜₒₜ of ISF with n molecular species capable of generating current when oxidized can be described by Equation 2:

$${I}_{tot} = {\sum }_{1}^{n} {I}_{i} = f ( {V}_{WE} , {c}_{1} , {c}_{2} , {c}_{3} , … {c}_{n} ) \qquad Equation 2$$

where cᵢ is the concentration of the ith molecular species.

As a simple example, for an ISF measurement where there are only two molecular species X and Y generating current due to oxidation at the working electrode, measured current Iₜₒₜ may be equal to (I_{X}+I_{Y}). For example, for an the ISF measurement where the only two molecular species generating current due to oxidation at the working electrode include glucose and acetaminophen, measured current Iₜₒₜ may be equal to (I_{glu}+I_{ace}).

The measurement mode discussed herein allows for deconvolution of Iₜₒₜ to estimate one or more currents Iᵢ due to an individual molecular species. As the sensor steps through potentials V_{ZE 1-m} of the interference zapping layer, certain molecular species may be oxidized before reaching the working electrode, such that they contribute no current to measurements taken by the working electrode. Molecular species X may have a higher oxidation potential than molecular species Y. In a simple example where Iₜₒₜ=(I_{X}+I_{Y}) when V_{ZE} is set to 0 V, when V_{ZE} is set to oxidize molecular species Y but not oxidize molecular species X, h at may be approximately equal to I_{X} alone, assuming I_{Y} is approximately 0: Iₜₒₜ=(I_{X}+0)=I_{X}. Generally, Iₜₒₜ may depend on fewer molecular species as V_{ZE} increases.

A hardware processor in electronic communication with a sensor in accordance with the present disclosure may be capable of comparing Iₜₒₜ at two or more zapping layer potentials V_{ZE} to determine the current contribution of one or more molecular species that were sensed by the working electrode. For example, again with reference to a simple example where Iₜₒₜ=(I_{X}+I_{Y}) when V_{ZE} is set to 0 V, a first zapping layer potential V_{ZE} A may be insufficiently high to oxidize molecular species Y, but a second zapping layer potential V_{ZE B} may be sufficiently high to oxidize molecular species Y but not sufficiently high to oxidize molecular species X. At V_{ZE A}, the working electrode can sense I_{tot A}, which may be equal to (I_{X}+I_{Y}). At V_{ZE B}, the working electrode can sense I_{tot B}, which may be equal to I_{X}, because molecular species Y may be prevented or inhibited from reaching the working electrode due to the potential V_{ZE B} of the interference zapping layer. A hardware processor in accordance with the present disclosure can subtract I_{tot B} from I_{tot A} to calculate Iv:

I_{tot A}−I_{tot B}=(I_{X}+I_{Y})−(I_{X})=I_{Y} Equation 3

A hardware processor can estimate the concentration of X based at least in part on the magnitude of current I_{X}. A hardware processor can estimate the concentration of Y based at least in part on the magnitude of current I_{Y}.

For ISF having a more than two molecular species, it may be desirable to measure current Iₜₒₜ at a plurality of interference zapping layer potentials V_{ZE} such that there are sufficient currents Iₜₒₜ for comparison. For instance, for an ISF having three species X, Y, and Z, where X has a higher oxidation potential than that of either of Y and Z, and Y has a higher oxidation potential than Z, it may be desirable to measure currents I_{tot C} corresponding to a zapping layer potential V_{ZE} insufficient to oxidize any of X, Y, and Z, I_{tot D} corresponding to a zapping layer potential V_{ZE} sufficient to oxidize Z but insufficient to oxidize X or Y, and I_{tot E} corresponding to a zapping layer potential V_{ZE} sufficient to oxidize Z and Y but insufficient to oxidize X. Thus,

I_{tot E}−I_{tot D}=(I_{X}+I_{Y}+I_{Z})−(I_{X}+I_{Y})=I_{Z} Equation 4

I_{tot D}−I_{tot C}=(I_{X}+I_{Y}+0)−(I_{X}+0)=I_{Y} Equation 5

I_{tot C}=I_{X} Equation 6

A hardware processor can estimate the concentration of X based at least in part on the magnitude of current I_{X}. A hardware processor can estimate the concentration of Y based at least in part on the magnitude of current I_{Y}. A hardware processor can estimate the concentration of Z based at least in part on the magnitude of current I_{Z}.

The approach discussed above may be generalized for ISF having more than three molecular species, for example by making more measurements at additional zapping layer potentials V_{ZE}. Additionally or alternatively, this approach may adapted for determining a current Iₜₒₜ of a group of molecular species having similar oxidation potentials. For example, stepping from a first zapping layer potential to a second zapping layer potential may eliminate or reduce current contributions to Iₜₒₜ by molecular species having an oxidation potential lower between the first zapping layer potential and the second zapping layer potential. For examples where the individual concentrations of those molecular species are not of interest, it may be undesirable to set the zapping layer potential to a value between the first zapping layer potential and the second zapping layer potential.

In some aspects, the measured current Iₜₒₜ may be continuous (e.g., measured over a continuous working electrode potential V_{WE}). In some aspects, the measured current Iₜₒₜ may include discrete measurements. For example, again with reference to FIG. 7, Iₜₒₜ may include the set of currents measured at a single interference zapping layer potential V_{ZE} For example, I_{tot 1} measured at V_{ZE 1} may include (I_{1,1}, I_{2,1}, I_{3,1}, I_{4,1}, . . . I_{n,1}); I_{tot 2} measured at V_{ZE 2} may include (I_{1,2}, I_{2,2}, I_{3,2}, I_{4,2}, . . . I_{n,2}); I_{tot 3} measured at V_{ZE 3} may include (I_{1,3}, I_{2,3}, I_{3,3}, I_{4,3}, . . . I_{n,3}); I_{tot 4} measured at V_{ZE 4} may include (I_{1,4}, I_{2,4}, I_{3,4}, I_{4,4}, . . . I_{n,4}); I_{tot m} measured at V_{ZE m} may include (I_{1,m}, I_{2,m}, I_{3,m}, I_{4,m}, . . . I_{n,m}). The hardware processor may be capable of fitting a curve to Iₜₒₜ in aspects where Iₜₒₜ includes discrete measurements.

### Terminology

As used herein, “molecular species” refers to one or more types of molecule that may be sensed by a working electrode. A “molecular species” may be an analyte or an electrochemical interferent.

“Interference zapping layer,” “zapping layer,” “interference layer,” “interfering zapping layer,” and “interfering layer” are all used interchangeably herein.

A “working electrode” is an electrode of an electrochemical sensor on which a reaction of interest may occur. The terms “working electrode” and “sensing electrode” are used interchangeably herein.

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid-state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. An electrochemical probe comprising:
an electrode;
an enzyme layer; and
an interference zapping layer.

2. The probe of claim 1, further comprising an analyte limiting layer.

3. The probe of claim 2, wherein the analyte limiting layer is a glucose limiting layer.

4. The probe of claim 1, wherein the electrode is configured to measure glucose concentration.

5. The probe of claim 1, wherein the enzyme layer is configured to convert glucose to hydrogen peroxide and gluconic acid.

6. The probe of claim 5, wherein the enzyme layer comprises a glucose oxidase.

7. The probe of claim 1, further comprising a blocking layer.

8. The probe of claim 1, comprising:
a first voltage source configured to set the electrode to a first applied potential, and
a second voltage source configured to set the interference zapping layer to a second applied potential,
wherein the second applied potential is equal to or higher than the first applied potential.

9. The probe of claim 1, the interference zapping layer comprising a hydrogel.

10. The probe of claim 1, wherein the interference zapping layer comprises a microwire or nanowire.

11. A method of using an electrochemical probe, comprising:
applying a first potential to an interference layer, the first potential sufficient to oxidize at least one electrochemical interferent and a molecule of interest;
measuring a first background current of a first electrode;
measuring a second background current of a second electrode;
applying a second potential to the interference layer, the second potential sufficient to oxidize at least one electrochemical interferent but not oxidize the molecule of interest;
measuring a first current of the first electrode;
measuring a second current of the second electrode;
determining an estimate of a concentration of the molecule of interest based, at least in part, on measurements of the first background current, the second background current, the first current, and the second current.

12. The method of claim 11, the molecule of interest comprising glucose.

13. The method of claim 11, wherein the first potential is within +0.5 to +1.5 V.

14. The method of claim 13, wherein the first potential is within +0.6 to +1.1V.

15. The method of claim 11, wherein the second potential is within +0.3 to +1.1V.

16. The method of claim 15, wherein the second potential is within +0.4 to 0.7V.

17. A method of using an electrochemical probe comprising an interference zapping layer and a working electrode, the method comprising:
applying a first plurality of potentials to the interference zapping layer;
applying a second plurality of potentials to the working electrode;
measuring a plurality of currents of the working electrode, each of the plurality of currents measured while the interference zapping layer is set to one of the first plurality of potentials and while the working electrode is set to one of the second plurality of potentials; and
determining, with a hardware processor, an estimate of a concentration of an analyte based, at least in part, on the measured plurality of currents.

18. The method of claim 17, wherein applying the first plurality of potentials to the interference layer comprises sequentially applying the first plurality of potentials.

19. The method of claim 17, wherein applying the second plurality of potentials to the working electrode comprises sequentially applying the second plurality of potentials.

20. The method of claim 17, wherein each of the plurality of currents is measured at a different combination of one of the first plurality of potentials and one of the second plurality of potentials.
